(19) United States
(12) Patent Application Publication
(10) Pub. No.: US 2026/0334804 A1
(43) Pub. Date: Oct. 29, 2026

(54) AUGMENTING ACCURACY OF WEARABLE DEVICE MEASUREMENTS

(71) Applicant: VITAL SIGNALS, INC., Los Altos, CA (US)

(72) Inventors: Tom MOSS, Los Altos, CA (US); Mohammad USMAN, Mission Viejo, CA (US); Faisal KASHIF, Irvine, CA (US)

(21) Appl. No.: 19/652,439

(22) Filed: Apr. 20, 2026

Related U.S. Application Data
(60) Provisional application No. 63/795,734, filed on Apr. 28, 2025.
(60) Provisional application No. 63/837,897, filed on Jul. 3, 2025.

(51) Int. Cl. A61B 5/024; A61B 5/00; A61B 5/0245; A61B 5/11; G06F 3/01; G06F 3/04817

(52) U.S. Cl. CPC A61B 5/02416; A61B 5/0245; A61B 5/1102; A61B 5/7282; G06F 3/011; G06F 3/04817

(57) ABSTRACT

Examples include one or more processors that receive an indication to take a blood pressure measurement of a user of a wearable device. The processor(s) receives photoplethysmographic (PPG) sensor data from the wearable device. The processor(s) further receives at least one of other sensor data or other information indicative of at least one of an environment of the user, a current or past activity of the user, or a bodily condition of the user. The processor(s) inputs the received PPG sensor data and the at least one of the other sensor data or the other information into at least one computational model configured to output a blood pressure measurement and an associated confidence score based at least on the received PPG sensor data and the at least one of the other sensor data or the other information. In some examples, context information is determined and associated with the measurement result.

### CROSS-REFERENCES TO RELATED APPLICATIONS

This application claims the benefit of U.S. Provisional Patent Application No. 63/837,897, filed Jul. 3, 2025, and U.S. Provisional Patent Application No. 63/795,734, filed Apr. 28, 2025, which are incorporated by reference herein. Additionally, U.S. patent application Ser. No. 18/937,625, filed on Nov. 5, 2024, is incorporated by reference herein.

### BACKGROUND

Detecting body signals for determining and monitoring various vital signs and other bodily conditions of an individual can provide an indication of the health and wellbeing of the individual. Examples of such bodily conditions can include heart rate, blood pressure, body temperature, respiratory rate, blood oxygen saturation, heart rate variability, sleep tracking, blood sugar, and various other physiological characteristics of the individual.

Recently, wearable devices have become popular for monitoring vital signs and other bodily conditions. However, conventional devices and techniques are not always able to achieve suitable results. For example, accurately measuring blood pressure may typically require an inflatable cuff to be placed on an extremity of the individual whose blood pressure is being measured. While the use of an inflatable cuff can usually provide an accurate measurement of an individual's blood pressure, this measurement technique is not practical for frequent and non-intrusive measurement of blood pressure, such as while the individual is going about daily activities, sleeping, exercising, or the like.

### BRIEF DESCRIPTION OF THE DRAWINGS

The detailed description is set forth with reference to the accompanying figures. In the figures, the leftmost digit(s) of a reference number identifies the figure in which the reference number first appears. The use of the same reference numbers in different figures indicates similar or identical items or features.

FIG. 1 illustrates an example system for measuring and monitoring bodily conditions according to some implementations.

FIG. 2 illustrates an example wearable device that can be used for measuring and monitoring bodily conditions according to some implementations.

FIG. 3 illustrates an example of an insert that may be used for reducing an inner dimension of the wearable device according to some implementations.

FIG. 4 illustrates an example user interface that may be presented on the user device for providing interactive real-time instructions to the user when taking a spot measurement of a bodily condition according to some implementations.

FIG. 5 illustrates the example user interface that may be presented on the user device for providing interactive real-time instructions to the user when taking a spot measurement of a bodily condition according to some implementations.

FIG. 6 illustrates the example user interface that may be presented on the user device for providing interactive real-time instructions to the user when taking a spot measurement of a bodily condition according to some implementations.

FIG. 7 is a flow diagram illustrating an example process for providing interactive real-time instruction to a user for performing a spot measurement according to some implementations.

FIG. 8A illustrates select example components of the user device and the wearable device that may be used to implement at least some of the functionality described herein according to some implementations.

FIG. 8B illustrates select example components of a wearable device that may implement at least some of the functionality described herein according to some implementations.

FIG. 8C illustrates select example components of the service computing device(s), the user device, and the wearable device that may be used to implement at least some of the functionality herein according to some implementations.

FIG. 9 illustrates an example user interface for presenting a plurality of blood pressure measurements over time with estimated error ranges according to some implementations.

FIG. 10 illustrates an example of using a computational model configured as an optimized joint model for determining a blood pressure measurement and confidence score according to some implementations.

FIG. 11 illustrates an example of using a plurality of optimized dedicated models and an optimized joint model for determining an estimated blood pressure measurement and an associated confidence score according to some implementations.

FIG. 12 illustrates an example user interface that may be presented on the user device for providing blood pressure measurement information to the user after taking a spot blood pressure measurement according to some implementations.

FIG. 13 illustrates an example process for determining a blood pressure measurement and confidence score according to some implementations.

FIG. 14 illustrates an example of using a computational model configured as an optimized joint model for determining a blood pressure measurement based on a first set of sensor data and/or information, and determining a confidence score based on a second set of sensor data and/or information according to some implementations.

FIG. 15 illustrates an example of using one or more optimized dedicated models for determining an estimated blood pressure measurement based on a first set of sensor data and/or information, and determining a confidence score based on a second set of sensor data and/or information according to some implementations.

FIG. 16 illustrates an example user interface that may be presented on the user device before a user takes a blood pressure measurement according to some implementations.

FIG. 17 illustrates an example user interface that may be presented on the user device for receiving context information prior to beginning a blood pressure measurement according to some implementations.

FIG. 18 illustrates an example user interface that may be presented on the user device according to some implementations for entering a context factor not included in the listing of context factors.

FIG. 19 illustrates an example user interface that may be presented on the user device following completion of the blood pressure measurement according to some implementations.

FIG. 20 illustrates an example user interface that enables the user to filter blood pressure measurement results based on associated context factors according to some implementations.

FIG. 21 is a flow diagram illustrating an example process for associating and considering context information with blood pressure measurements according to some implementations.

### DETAILED DESCRIPTION

Some implementations include devices and techniques for detecting and monitoring vital signs and other bodily conditions of a user. For instance, at least one wearable device may be worn by the user of the wearable device. The wearable device may include one or more sensors able to detect bodily signals of the user, such as for determining one or more bodily conditions of the user. The wearable device may be used for taking “spot” measurements of one or more bodily conditions and which may be performed by the user periodically. The wearable device may also be used for continuous measurement and monitoring of the one or more bodily conditions of the user over a longer term, such as by performing the continuous measurement and monitoring while the user goes about their daily routine, eats, exercises, sleeps, or the like.

In some examples, the wearable device may be used, at least in part, for measuring the blood pressure of a user. Further, the accuracy of the blood pressure measurements taken using the wearable device may be augmented based at least in part on receiving other sensor data from other devices and/or based on receiving other information related to the user, such as the user's activities and/or the user's environment. Additionally, user context information may also be received and applied for providing insight regarding various context factors that may affect the user's blood pressure during measurement. For instance, the various context factors may include conditions or events of the user such as whether the user has exercised recently, consumed caffeine, taken blood pressure medication, eaten food, consumed alcohol, smoked, taken a cold plunge, is menstruating, and so forth. Further, in the case that one or more of these context factors do apply while a blood pressure measurement is being taken, the context factor can be associated with and stored with the blood pressure measurement to indicate a possible contributing factor of a higher-than-usual or lower-than-usual blood pressure measurement.

One or more computational models, such as neural networks or other machine-learning models, may be trained to receive numerous different types of information in addition to sensor information received from the wearable device. Some or all of this available sensor data and other information, including the factors listed above, may be used to determine a confidence score indicating a predicted accuracy of a blood pressure measurement. Additionally, in some cases, the computational model(s) may be trained to take into account some or all of the additional sensor data, application data, context information, and other information when determining the blood pressure measurement for outputting a more accurate blood pressure measurement and/or for determining an amount of confidence in the blood pressure measurement.

Additionally, the wearable device may be able to communicate with a user device associated with the user and/or able to communicate over a network with a network computing device, such as a server, cloud computing device, or the like. For example, a user device such as a smartphone, tablet, laptop, desktop, smart glasses (including augmented reality glasses), augmented reality headset, or any other computing device of the user may have an array of capabilities not possessed by the wearable device, including a screen that is larger than a screen of the wearable device, if any. Accordingly, the user device may enable the user to view a graphical user interface that can assist the user in taking spot measurements and also enable viewing results of spot measurements, as well as the results of the continuous measurements captured over time. For instance, the spot measurements of the bodily conditions may be viewed by the user and compared with baselines traditionally collected using various conventional vital sign measurement devices such as a cuff-style blood pressure monitor for detecting blood pressure, an ECG device for detecting heartbeat patterns, a thermometer for detecting body temperature, an oximeter for detecting blood-oxygen levels, and so forth.

On the other hand, traditional baselines for the continuous measurement results that may be collected while a user performs various different activities do not typically exist. Accordingly, the continuous measurement results may be viewed by the user for various additional purposes, such as for identifying any trends that may be apparent from the continuous measurements, for detecting any spikes in the measured values that might differ from the usual measurements collected in the past during the same activities, same times of day, etc., and/or for determining differences in the measured bodily conditions during different activities or situations, such as differences between measurements captured during sleeping, working out, relaxing, sitting, standing, working, driving, and so forth.

Additionally, one problem with users taking spot measurements on their own is that the spot measurements may often be taken under differing conditions, such as differing sensor placement relative to the user's body, differing body positions (e.g., standing, sitting, lying down) and so forth, that differ from when prior spot measurements were taken, and which can affect the usefulness of the spot measurement results due to inconsistencies in how the spot measurements are taken. To address this problem, examples herein may provide interactive instructions to a user for causing the user to take the spot measurements under similar conditions each time a spot measurement for a particular bodily condition is taken. This helps to ensure consistency in at least some of the controllable external variables so that a comparison of spot measurement results can be made over time for determining possible changes in the bodily condition being measured, while minimizing the influence of the external variables that might otherwise affect the accuracy of the spot measurement of the bodily condition.

Furthermore, such as in the case of taking blood pressure measurements, various events or conditions of the user may affect the user's blood pressure by causing variations in the user's blood pressure. Thus, the current context of a user regarding recent events or conditions may be determined for indicating whether the blood pressure of the user may have been affected. As mentioned above, examples of such events or conditions may include whether the user has recently consumed caffeine, alcohol, food, etc., whether the user has smoked recently, has exercised recently, taken blood pressure medication, and so forth. As discussed additionally below, in some examples, these context factors may be detected automatically and included as information that may be provided to the one or more machine learning models herein. Further, in some examples, the user may manually enter context information both before and after the blood pressure measurement is taken.

To assist the user in taking consistent spot measurements, an application executing on the user device may provide the user with interactive real-time guidance when taking a measurement. As one example, in the case of taking a blood pressure spot measurement, the user application may initially instruct the user to direct a camera of the user device toward the user. For instance, in the case of a smartphone or tablet, the user may direct the front camera toward the user so that the user is concurrently able to view a screen of the user device with an image of the user presented thereon. Similarly, in the case that the user device is a laptop or desktop computer, the user may be instructed to sit in front of a webcam or other camera associated with the user device so that the user is also able to view a screen of the user device. As another example, in the case that the user device includes smart glasses or an augmented reality headset worn by the user, and that includes a camera and virtual screen, the user may be instructed to sit in front of a mirror so that the camera can capture an image of the user's reflection and display the image on the virtual screen provided by the glasses or headset, or in another example, view the reflection of the user with an augmented reality overlay.

In some examples, the user application may employ a computational model, such as to check the user's body position (e.g., sitting, standing, etc.) and the location of the wearable device relative to the user's body. For example, the computational model may determine whether the wearable device is positioned at an optimal location relative to the user's body. In particular, for the example in which a spot measurement of the user's blood pressure is to be taken, the computational model may check whether the wearable device is close to being level with the user's heart, whether the user's hand is resting palm up and supported by a surface, and so forth.

If the user application detects that the user is, for example, standing instead of sitting, or the user has the wearable device in a non-optimal location relative to the user's body, the user application may provide instructions and/or an interactive animation overlaid on the image of the user, such as to show the user where to position the wearable device, how to position the user's body, or the like. For instance, prior to taking a spot measurement of blood pressure, the user application executing on the user device may prompt the user to activate the camera on the user device and direct the camera in the direction of the user so that an image of the user can be captured and evaluated by the user application via the computational model. Additionally, a live real-time image of the user may be presented to the user in a graphical user interface such that the user may see the user's body position and wearable device position. In some examples, the user application may overlay graphics, animations, instructions, or the like onto the image of the user to help instruct the user for achieving an optimal position for measuring the bodily condition. As another example, the user application may retrieve one or more images from a past spot measurement that was performed correctly for the same bodily condition and may present some or all the images for showing the user how to take the spot measurement.

The user application may further provide additional instructions to the user once an optimal position of the user's body and the wearable device has been achieved. For instance, in the case of measuring blood pressure, the user application may instruct the user to inhale, subsequently exhale, and then relax, breathe normally, and hold still while the blood pressure measurement is taken. In addition, the user application may display a timer countdown to let the user know how much longer to hold still until the blood pressure measurement is completed.

In some examples, the user application may refer to information retained from one or more past spot measurements taken by the user for the same bodily condition for ensuring that the user takes the spot measurements consistently for the same bodily condition and under similar conditions. For instance, when the user first takes a spot measurement for a particular bodily condition, the user application may provide instructions to the user for taking the spot measurement with optimal positioning of the user and the wearable device for the particular bodily condition being measured. Additionally, the user application may instruct the user to activate the camera on the user device to capture video of the user when taking the spot measurement. For subsequent spot measurements for the same bodily condition, the computational model may access one or more images captured during one or more past spot measurements to use as a reference for comparing a current position of the user's body and the wearable device relative to the user's body, such as for instructing the user on how best to achieve an optimal position of the user's body and the wearable device for taking the spot measurement.

Further, in other examples, the user application might not have the benefit of a video feed of the user during the taking of the spot measurement, such as if the user does not activate the camera, the user device does not have a suitable camera, or the like. In this situation, the user application may still present instructions to the user regarding how to position the user's body and how to position the wearable device, how to breath or perform other actions, etc. However, the user application may not be able to provide additional interactive instructions to the user in real time without receiving additional information about the user's responses to the instructions.

In some examples, the wearable device herein may be, or may include, a ring that is worn on a finger of the user. In other examples, the wearable device may be, or may include, any of a watch, bracelet, wristband, armband, fingertip clip, glove, chest strap, or other configurations, as additionally listed below. The wearable device may provide at least one of sensor data or a determined bodily condition value to the user device or other computing devices, depending on the processing capabilities and configuration of the wearable device.

Some examples herein may include one or more wearable devices having one or more sensors configured to measure bodily signals of a user's body. In some examples, multiple sensing modalities may be employed, such as via one or more of a photoplethysmographic (PPG) sensor, an electrocardiogram (ECG) sensor, a ballistocardiogram (BCG) sensor, an inertial measurement unit (IMU), a microphone (MIC) and/or other sensors, as discussed additionally below. In some examples, the BCG sensor may be an accelerometer. In other examples, the IMU may also serve as the BCG sensor while also having the ability to detect other physiological functions such as breathing, activity, or position of the individual. Techniques and arrangements for detecting body signals for measuring and monitoring bodily conditions according to some examples herein are described in U.S. patent application Ser. No. 18/937,625, filed on Nov. 5, 2024, and which has been incorporated by reference herein in its entirety.

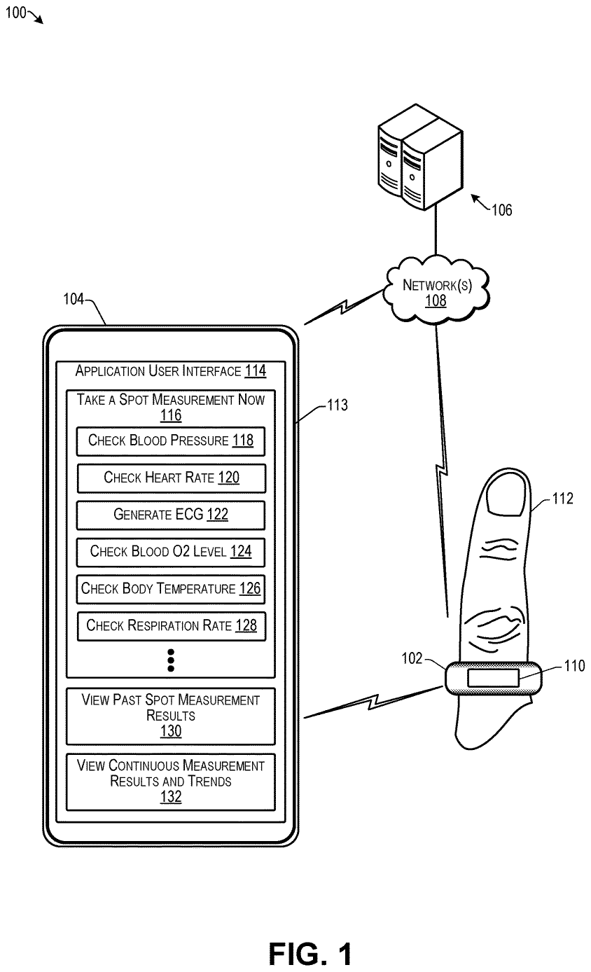

FIG. 1 illustrates an example system 100 for measuring and monitoring bodily conditions according to some implementations. In this example, a wearable device 102 may be worn by a user and may be in communication with a user device 104 and/or one or more network-located service computing devices 106. For example, the wearable device 102 may communicate with the user device 104 through a wireless connection or other suitable connection, such as via BLUETOOTH®, WiFi, or other short-range radio communication technology. Additionally, or alternatively, the wearable device 102 and/or the user device 104 may communicate over one or more networks 108 with the one or more service computing devices 106, which may include one or more cloud servers, one or more service computing devices, one or more local computing devices, or the like. Furthermore, while only one wearable device 102 is illustrated in this example, in other examples, there may be multiple wearable devices 102 worn by the user on various different locations of the user's body. For example, a first wearable device 102 may have one or more first sensors and a second wearable device 102 may have one or more second sensors that may be the same as or different from the sensors on the first wearable device 102. In some cases, different types of sensors at different body locations may be used for measuring different bodily conditions.

The one or more networks 108 may include any suitable type of communication network enabling wireless and/or wired communications, and may include local area networks, wide area networks, the Internet, virtual private networks, cellular networks, WiFi networks, short-range radio communications, such as BLUETOOTH®, and so forth.

In some cases, the wearable device 102 may include at least one of a human-machine interface for communicating information to a human, and/or an electronic communication capability for communicating electronically with other computing devices. In some examples, the wearable device 102 may include human-machine interfaces such as a display 110, and/or a speaker, one or more buttons, controls, or other input devices (not shown in FIG. 1). The wearable device 102 may further include one or more sensors (not visible in FIG. 1) that may detect bodily signals from the user when the wearable device 102 is worn by the user, such as on a finger 112, or other body part of the user. In some examples, the wearable device 102 may include at least one processor for at least one of storing, transmitting, or processing the sensor data collected by the one or more sensors.

In some examples, the wearable device 102 may include sufficient processing capacity for determining bodily condition measurements from the received sensor data. In other examples, the wearable device 102 may merely buffer and transmit the received sensor data to the user device 104 or the service computing devices 106, and the receiving device 104 or 106 may perform the processing of the sensor data for determining the bodily condition measurement. In yet other examples, some of the bodily condition measurements may be determined onboard the wearable device 102, while other bodily condition measurements that utilize higher processing capacity may be determined onboard the user device 104 or the service computing devices 106. Numerous other variations will be apparent to those of skill in the art having the benefit of the disclosure herein.

In some examples, the user device 104 may have a display 113, and may be, or may include, a smartphone, tablet, or the like. In other examples, the user device 104 may be or may include a laptop, desktop, smart glasses, augmented reality glasses, augmented reality headset, or other computing device accessible by the user and able to communicate with the wearable device 102. The user device 104 may execute a user application (not shown in FIG. 1) able to instruct and assist the user in taking spot measurements of various bodily conditions and also able to determine a bodily condition from received sensor data. In the illustrated example, the user application presents an application user interface 114, which may include a graphical user interface in some examples. As indicated at 116, the user interface 114 may enable the user to select one or more of a plurality of bodily conditions for taking a spot measurement of a selected bodily condition. Examples of spot measurements of bodily conditions that may be selected include checking blood pressure 118, checking heart rate 120, generating an electrocardiogram 122, checking blood oxygen level 124, checking body temperature 126, checking respiration rate 128, and so forth. Examples of additional bodily conditions that may be checked, measured, tracked, monitored, or the like, according to implementations herein may include heart rate variability, sleep duration and stages, circadian rhythms, blood sugar, ovulation and menstrual cycles, pregnancy, stress levels, exercise activity, steps, cadence, calories burned, workout intensity, metabolism patterns, and so forth.

In addition, the user interface 116 may include a capability for the user to view past spot measurement results, as indicated at 130. For example, the user may select this control and may then select past spot measurement results for a selected bodily condition, such as any of those enumerated above.

Further, as indicated at 132, the user may also use the user interface 116 for viewing continuous measurement results and trends. As mentioned above, the continuous measurements of the various bodily conditions may not have the same clinical significance as the spot measurements, but instead may be used for identifying trends that might be determined from the continuous measurements. The continuous measurements may be used for detecting any spikes in the measured values that might differ from the usual measurements collected in the past during the same activities, same times of day, etc., and/or for determining differences in the measured bodily conditions during different activities or situations, such as differences between measurements captured during sleeping, working out, relaxing, sitting, standing, working, driving, and so forth.

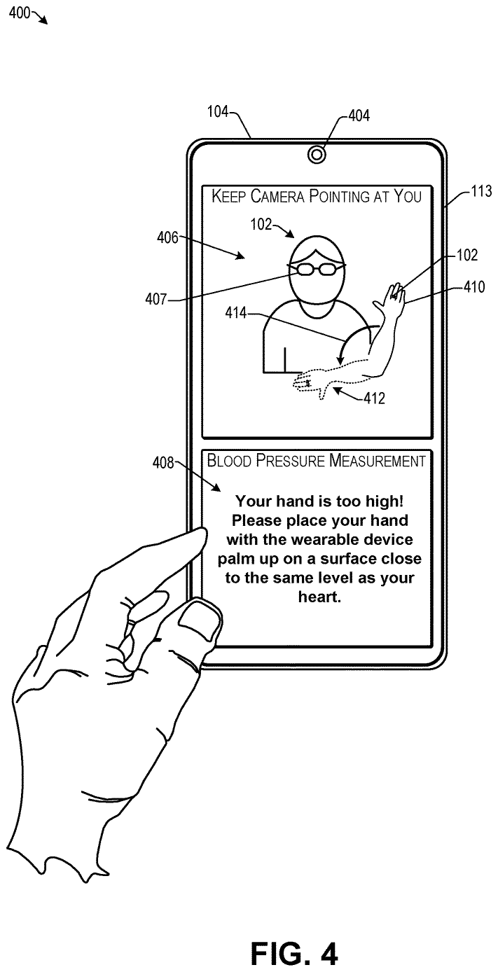

Additionally, the examples herein may employ one or more computational models. The computational models herein may include one or more of neural networks or other artificial intelligence models, machine-learning models, trainable models, mathematical models, statistical models, heuristic models, simulation models, combinations thereof, or any of various other types of models described elsewhere in this disclosure and that may be used to estimate, calculate, or otherwise assist in performing the functions described herein. For example, an instructional computational model may be used by the user application for instructing a user when taking a spot measurement of a bodily condition using the wearable device herein, such as described below with respect to FIGS. 4-7. In addition, one or more bodily condition computational models may be used by the user application on the user device (or in other cases, a wearable device application on the wearable device) for determining a measured value of a bodily condition based on receiving sensor data from one or more sensors configured to sense one or more bodily signals of a user that are indicative of the bodily condition.

Further, while some examples herein are described in the context of determining blood pressure measurements and other bodily conditions for people, the devices and techniques herein may be similarly applied to other animals. In some examples, the bodily condition computational model(s) may be obtained based on physiological principles and/or may be trained using machine-learning methods. As one example, at least one bodily condition computational model may be configured to provide an indication of blood pressure based on receiving an input of sensor data from one or more sensors, such as the PPG sensor discussed above.

In addition, some examples herein may take into account the specific anatomy and physiology of the user, including arterial stiffness and other physical conditions of the particular user. For instance, the bodily condition computational model(s) herein may account for arterial and venous characteristics such as resistance, compliance, and inductance, and may be based in part on physiology for fluid flow and pressure. On the other hand, many conventional systems ignore the differences in the vasculature conditions of individual users, which may vary significantly from one user to the next. In addition, some examples herein may take into account variations in the condition of each individual user over time, such as may be due to exercise regularity, medication, hydration levels, and so forth.

In some implementations, a single bodily condition computational model may be trained as a joint model that is able to receive inputs of sensor data from multiple different sensor types, such as PPG, ECG, BCG, sound, IMU, and so forth, for determining a one or more bodily condition measurements. For instance, the joint model may incorporate features and/or parameters for all of the different sensor types from which sensor data may be received for determining one or more bodily conditions, and may be optimized according to an estimation template that may be applied in one step on all of the inputted sensor data for determining a variety of bodily conditions using the single bodily condition computational model.

As another example, multiple bodily condition computational models may be trained, with each respective bodily condition computational model being trained or derived for a particular sensor type. Under this technique, the system may include an optimized PPG bodily condition computational model, an optimized ECG bodily condition computational model, an optimized BCG bodily condition computational model, and so forth. The outputs of the multiple bodily condition computational models may be aggregated, such as based on a bias versus estimator variance, for providing various bodily condition measurements.

In examples herein, some of the bodily conditions (e.g., blood pressure, heart rate, blood oxygen, etc.) may be measured and monitored continuously over time and presented to a user, such as on the display 113 of the user device 104, or other display accessible to the user. Additionally, spot measurements of at least some of the bodily conditions may be taken on demand, such as in response to a user input and/or request.

In some implementations, the one or more service computing devices 106 may send, e.g., over a network, to at least one of the user device 104 or the wearable device 102, one or more of the bodily condition computational models, such as AI models, machine-learning models, and the like, that are trained and used for determining bodily conditions from sensor data. The bodily condition computational model(s) may be trained by the service computing devices 106 to be used for determining bodily condition values based at least in part on sensor data received from the wearable device 104. Similarly, the instructional computational model may also be trained by the service computing devices 106 and provided to the user device 104 as an accompaniment to the user application that is executable on the user device for providing instructions to the user when taking a spot measurement of a bodily condition.

In other examples, one or more of the computational models may reside on one or more of the service computing devices 106. In this case, the user device 104 and/or the wearable device 102 may send sensor data and other information such as environment information, user information, context information, and so forth, to the service computing device 106. The service computing device 106 may input the received sensor information and other information into the one or more computational models for determining one or more bodily conditions of the user, such as blood pressure, heart rate, body temperature, respiratory rate, blood oxygen saturation, heart rate variability, blood sugar, and various other physiological characteristics. The service computing device 106 may further determine a confidence score associated with each measurement, and may indicate any context factors that may have influenced a measured bodily condition. The network computing device may send the measurement information, confidence score(s), context factor(s), and so forth to user device 104 and/or the wearable device 102 for storage and/or presentation by at least one of the user device 104 or the wearable device 102.

Examples of computational models that may be used in some examples herein may include neural networks such as deep learning models, recurrent neural networks, LSTM models, convolutional neural networks, feedforward neural networks, and so forth, to name a few. Further, other types of machine learning and statistical models may be used, such as models trained using supervised learning algorithms (e.g., Bayesian statistics, support vector machines, decision trees, classifiers, k-nearest neighbor, etc.), unsupervised learning algorithms (e.g., association rule learning, hierarchical clustering, cluster analysis, etc.), semi-supervised learning algorithms, and so forth. Implementations herein are not limited to any particular type of machine learning or otherwise trainable model, as well as the other examples described elsewhere herein. Further, in some examples, one or more heuristic models and/or mathematical models, or the like, may be used to perform some or all of the functions described herein in place of one or more machine learning models. Additionally, some examples may employ a combination of machine-learning models, heuristic models, and/or mathematical models.

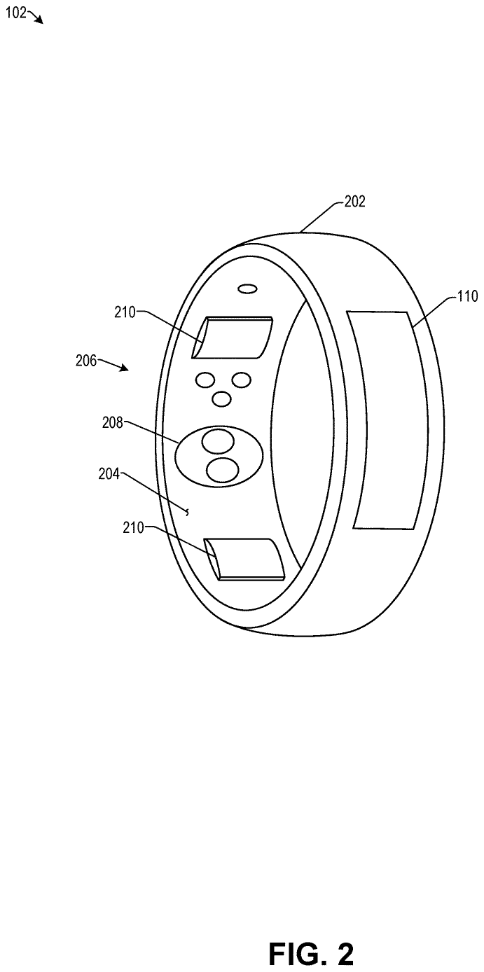

FIG. 2 illustrates an example wearable device 102 that can be used for measuring and monitoring bodily conditions according to some implementations. The wearable device 102 in this example may include a durable outer surface 202, which in some examples, may include the display 110, and an inner surface 204 having one or more sensors 206 included thereon. At least some of the one or more sensors 206 may be positioned for facing and/or coming into contact with the skin of a user when the user is wearing the wearable device. In the illustrated example, the wearable device 102 is embodied as a ring that may be finger-mounted, such as worn on a finger or thumb of a user. In other examples, however, the wearable device 102 may be any of: a different type of finger-mountable device, such as a fingertip-worn device, artificial fingernail, multi-knuckle ring, or the like; a hand-mountable device, such as a glove; a wrist-mountable device, such as a watch, bracelet, or the like, worn on the wrist of the person; an arm-mountable device, such as a sleeve, strap, band, patch, or the like, mounted on the upper arm or forearm of the person; a chest-mountable device, such as a patch, strap, belt, harness, or the like, mounted on the chest of the person, a foot-mountable device, leg mountable device, ear-mountable device, head-mountable device, neck-mountable device, and so forth. Further, while several types of wearable devices are described in the examples herein, other types of wearable and body-mountable devices may alternatively be used, as will be apparent to those of skill in the art having the benefit of the disclosure herein.

In some examples, the wearable device 102 may include at least one PPG sensor that may be used for detecting at least the blood pressure, heart rate, and/or blood oxygen levels of a user of the wearable device 102. For example, one or more photodiodes or other optical sensors 208 and one or more LEDs or other light sources 210 may be located on the inner surface 204 around a portion of the inner circumference of the wearable device 102 for serving as a PPG sensor. The light sources 210 may each generate a beam of light that is detected by the optical sensor 208, such as by reflecting off a body part of interest (e.g., an artery) or by passing through, or partially through, a body part of interest.

Furthermore, in some examples, the PPG sensor herein may be operated at a sampling rate that is substantially higher than conventional PPG sensors, such as at a sampling rate between 1000 and 2000 Hz, or even higher, which provides for high-fidelity detection of fluctuations in blood vessel diameters, locations, and so forth. This enables provision of a higher fidelity waveform of this information as the sensor data input signal, which provides for a more accurate measurement of the bodily condition, such as blood pressure, or other bodily condition.

In some examples, the sensors 206 may include one or more other sensors in addition to the PPG sensor, such as an electrocardiogram (ECG) sensor, a ballistocardiogram (BCG) sensor, a microphone, IMU, and/or other sensors. In some cases, use of the bodily condition computational model(s) herein for determining bodily condition measurements may enable a reduction in the number and types of sensors used. Further, some examples may include a computational model for accounting for unknown parameters of the vascular system, such as arterial resistance, compliance, and elastance.

The wearable device 102 may further include additional components such as a wireless communication interface to enable communication with the user device 104, such as through BLUETOOTH® or other short range radio signals, a battery (not shown), and various other components depending on the desired configuration and functionality of the wearable device 102. Additionally, in some examples, the wearable device 102 may include at least one processor and a memory or other computer-readable medium (not shown in FIG. 2), and may be able to perform processing of the received sensor data received from the sensors 206 for providing measured bodily condition values based on the sensor data collected by the sensors 206.

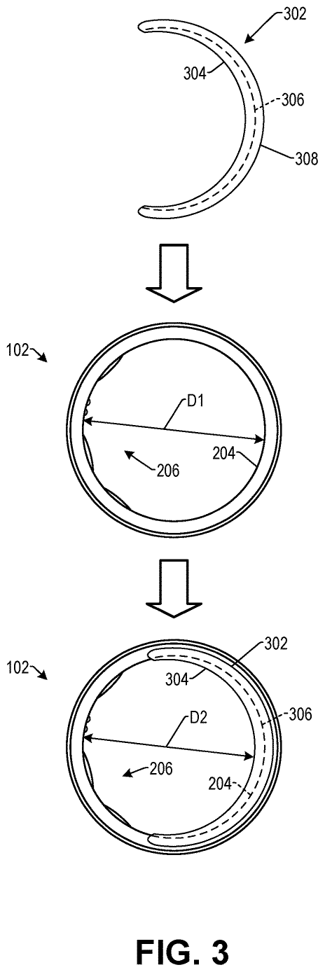

FIG. 3 illustrates an example of an insert 302 that may be used for reducing an inner dimension of the wearable device 102 according to some implementations. Some users may have a malady that causes their fingers to swell on occasion and return to a normal size thereafter. To accommodate these changes in the diameter of the user's finger, the wearable device 102 may include the insert 302 when the user's finger size is normal size and the insert 302 may be removed when the user's finger is swollen. For example, to obtain accurate measurement of certain bodily conditions, it is desirable for the sensors 206 to be snuggly in contact with the user's finger, but not overly tight. Further, the insert 302 is configured to avoid covering any portion of the sensors 206 when the insert 302 is inserted into the wearable device 102, thereby avoiding any impairment of the functionality of the sensors 206.

The insert 302 includes an inner surface 304 that contacts the user's finger when the insert 302 is inserted into the wearable device 102, and an outer surface 306 that contacts the inner surface 204 of the wearable device 102 when the insert 302 is inserted into the wearable device 102. Additionally, the insert 302 includes a pair of flanges 308 that engage either side of the wearable device 102 for helping to hold the insert 302 inside the wearable device 102 when the insert 302 is inserted into the wearable device 102. Furthermore, the insert 302 may include one or more locking features such as one or more pins, ridges, slots, or the like that engage with a respective complementary feature (not shown in FIG. 3) on the wearable device 102 when the insert 302 is inserted in the wearable device 102 to hold the insert 302 in a fixed position within the wearable device 102 with respect to the sensors 206 to avoid any coverage of the sensors 206.

In the example of FIG. 3, prior to inserting the insert 302 into the wearable device 102, the wearable device 102 may have a first inner diameter D1. After the insert 302 is inserted into the wearable device 102, the wearable device 102 may have an inner distance D2, that is smaller than the original inner diameter D1. Accordingly, a user may wear the wearable device 102 with the insert 302 in place when the user's finger is not swollen, and may remove the insert 302 when the user's finger swells to a point at which the wearable device 102 with the insert 302 in place is no longer comfortable or is overly tight. Furthermore, while one example of an insert 302 has been illustrated and described herein, numerous variations will be apparent to those of skill in the art having the benefit of the disclosure herein.

FIG. 4 illustrates an example user interface 400 that may be presented on the user device 104 for providing interactive real-time instructions to the user when taking a spot measurement of a bodily condition according to some implementations. The user interface 400 may be generated by the user application executing on the user device 104 when the user selects a virtual control for taking a spot measurement for a bodily condition, such as discussed above with respect to FIG. 1. In some examples, the user application may prompt the user to direct a front camera 404 or other camera associated with the user device 104 toward the user, such as on a wide-angle setting if available, so that the user application is able to receive an image 406 of the user. Alternatively, the user may be wearing smart glasses 407 that have a video camera built in for capturing video in real time. For example, the user may sit in front of a mirror wearing the smart glasses. The video from the smart glasses 407 may be projected onto a screen provided by the smart glasses or onto a screen of another device of the user, such as a phone, tablet, computer, or the like. The user application may present the image 406 of the user in the user interface 400 along with instructions for the user to keep the camera pointing in the user's direction. The user interface may further present written instructions to the user as indicated at 408, and/or may speak the instructions to the user using a text-to-voice function or the like.

In the illustrated example, suppose that the spot measurement requested by the user is for measuring the blood pressure of the user, with it being understood that spot measurement of other bodily conditions may be similarly guided by the user application. In some examples, the user application may employ the instructional computational model that may check the user's body position (e.g., sitting, standing, etc.) and the location of the wearable device 102 relative to the user's body. For example, the instructional computational model may receive the image 406 of the user as an input for determining whether the user's body position is optimal and for determining whether the position of the wearable device 102 with respect to the user's body is optimal for the desired spot measurement. For example, spot measurement of different bodily conditions may have different requirements for positioning of the wearable device and/or the user's body. In in the current example in which a spot measurement of the user's blood pressure is to be taken, the computational model may check whether the wearable device 102 is close to being level with the user's heart, whether the user's hand 410 with the wearable device 102 mounted thereon is resting palm up and supported by a surface, and so forth.

If the user application executing the instructional computational model detects that the user's body is not in an optimal position for measuring the bodily condition, for example, standing instead of sitting, or that the user has the wearable device 102 in a non-optimal position relative to the user's body, the user application may provide the instructions at 408, and/or may also provide an interactive animation 412 and/or instructional graphics 414 overlaid on the image 406 of the user. For instance, the animation 412 and or the instructional graphics 414 may show the user where to position the wearable device 102, how to position the user's body, or the like, or the like for achieving a position closer to an optimal position. As one example, the computational model may determine whether a current position of the user's body and/or a current position of the wearable device 102 is acceptable within a certain range of leeway that may be determined by the instructional computational model based at least in part on how much the accuracy of the spot measurement might be affected by movement of the wearable device 102 and/or the body of the user away from their respective optimal positions.

In this example, a live real-time image 406 of the user may be presented to the user in the user interface 400 such that the user may see the position of the user's body and the position of the wearable device 102. The user application may overlay, on the live image 406 of the user, graphics 414, such as one or more arrows or various other graphics to indicate to the user how to reposition the user's body and/or the wearable device 102. Additionally, or alternatively, the user application may overlay an animation or image 412 of an optimal position of the user's body and/or an optimal position of the wearable device 102. As another example, an image of the user's body position from a previous spot measurement (or e.g., just an image portion including the user's arm with the wearable device from the previous measurement) may be overlaid on, or interleaved with, the live image of the user in real time. Further, the instructions at 408 may also be presented to the user as text and/or read aloud to the user.

The instructions 408, the real-time animation or image 412 and/or the graphics 414 may all help instruct the user for achieving acceptable positions of the user's body and the wearable device 102 that are within a threshold range of the optimal body position and wearable device position for measuring the bodily condition. As one example, the user application may retrieve one or more images from a past spot measurement that was performed correctly for the same bodily condition and may present some or a portion of the past images overlaid on the live image 406 or interleaved with the live image 406 of the user for providing the animation or image 412 of the optimal positions for showing the user how to take the current spot measurement for consistency with past spot measurements.

To enable the user application to refer to information retained from one or more past spot measurements taken by the user for the same bodily condition for ensuring that the user takes the spot measurements consistently for the same bodily condition and under similar conditions, the user application may retain one or more images of the past spot measurements. For instance, when the user first takes a spot measurement for a particular bodily condition, the user application may provide detailed instructions to the user for taking the spot measurement with optimal positioning of the user's body and the wearable device 102 for the particular bodily condition being measured. Additionally, the user application may instruct the user to activate the camera 404 on the user device 104 to capture video of the user when taking the spot measurement. For subsequent spot measurements for the same bodily condition, the instructional computational model may access one or more images and/or videos captured during one or more past spot measurements of the bodily condition to use as a reference for comparing a current position of the user's body and the wearable device 102, and may detect any differences outside the acceptable threshold range. Based on the detected differences, the user application may instruct the user in real time on how best to achieve an acceptable position of the user's body and an acceptable position of the wearable device 102 for taking the current spot measurement. Additionally, in some cases, a button or other virtual control (not shown in FIG. 4) may be included in the user interface 400 for overriding the instructions of the user application and proceeding with the spot measurement.

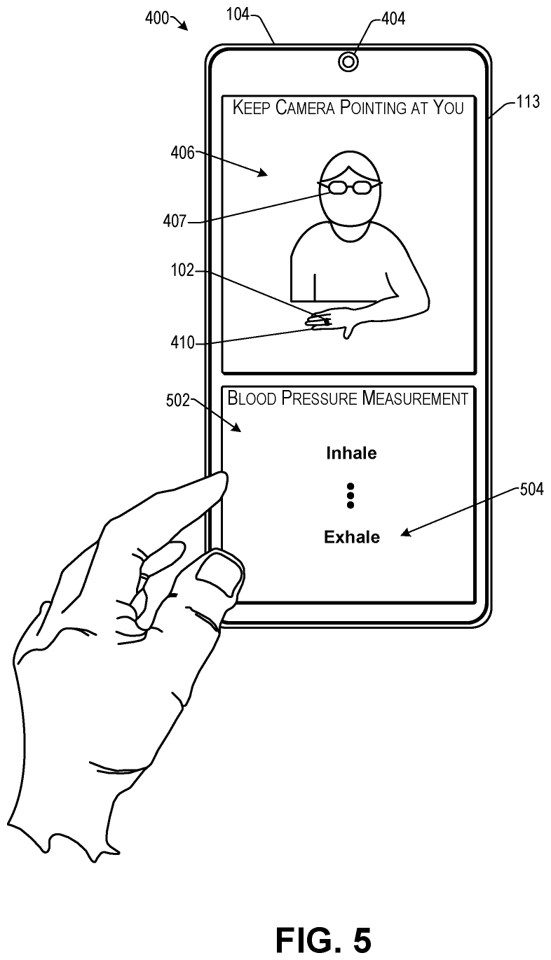

FIG. 5 illustrates the example user interface 400 that may be presented on the user device 104 for providing interactive real-time instructions to the user when taking a spot measurement of a bodily condition according to some implementations. In the example of FIG. 5, as a continuation of the example of FIG. 4, when the user application has determined that the user's body position and the position of the wearable device 102 are acceptable, the user application may provide additional real-time instructions to the user. For instance, in the case of measuring blood pressure, the user application may present a text instruction 502 for the user to inhale and may concurrently speak the instruction. The user application may wait while the user inhales, and subsequently present a text instruction 504 for the user to exhale and may concurrently speak the instruction. In some examples, the user device 104 may receive sensor data from the wearable device 102 for determining whether the user has completed inhaling before presenting the instruction 504 for the user to exhale. As another alternative, the instructional computational model may receive the live image 406 of the user as an input to determine when the user has inhaled before presenting the instruction to exhale.

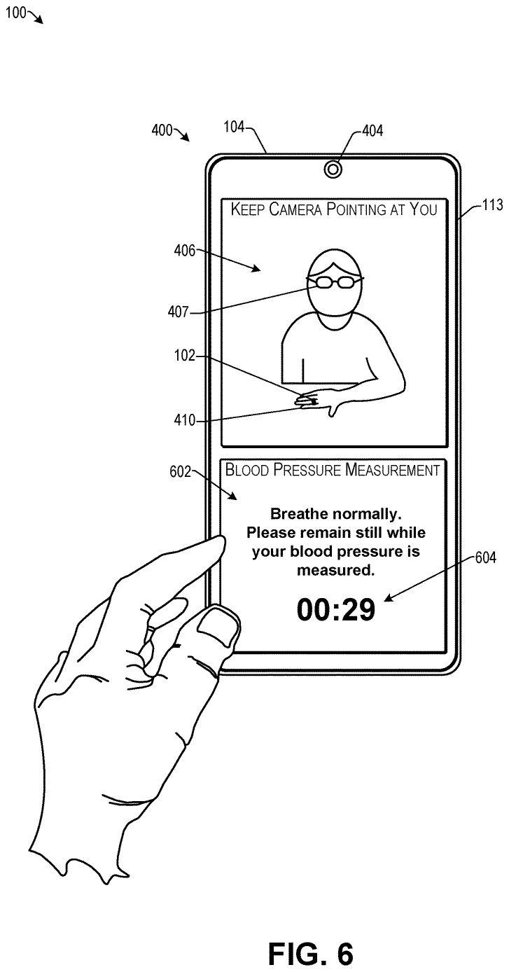

FIG. 6 illustrates the example user interface 400 that may be presented on the user device 104 for providing interactive real-time instructions to the user when taking a spot measurement of a bodily condition according to some implementations. In the example of FIG. 6, as a continuation of the example of FIGS. 4 and 5, when the user application has presented the instructions for the user to inhale and exhale, the user application may present text instructions 602 and concurrently may speak the instructions 602 for the user to breathe normally and remain still while the measurement is taken. In some cases, the instructions 602 are not presented until the user has completed exhaling, which may be detected using sensor data from the wearable device 102 and/or based on the live image 406 of the user.

In some examples, the user interface 400 may also present a countdown timer that indicates how much longer the user should remain still until the measurement is complete. Following completion of the measurement (e.g., when the countdown time reaches zero, the user interface may present an indication that the measurement is complete, and/or may announce that the measurement is complete. Further, the results of the measurement may also be presented (not shown in FIG. 6).

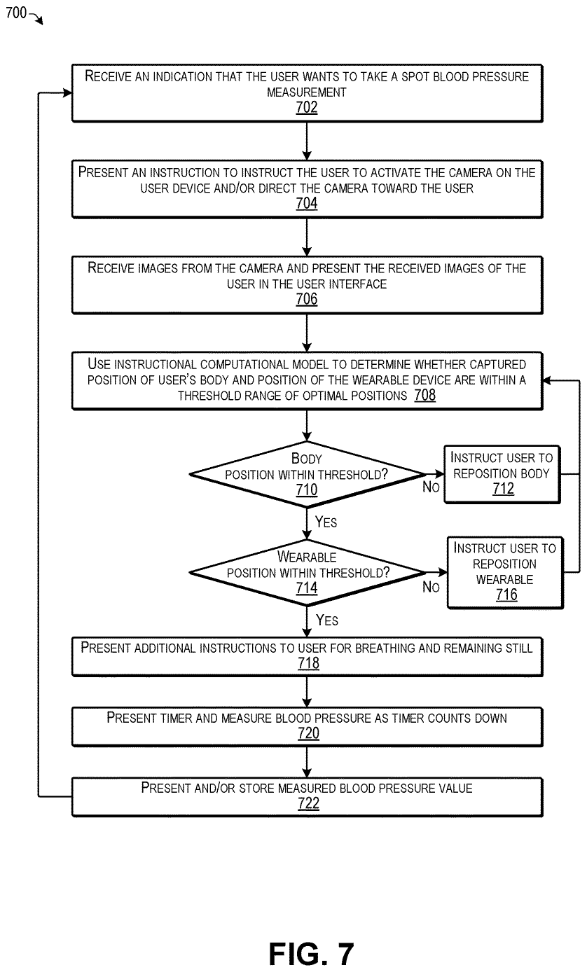

FIGS. 7, 13, and 21 include flow diagrams illustrating example processes according to some implementations. The processes are illustrated as collections of blocks in logical flow diagrams, which represent a sequence of operations, some or all of which can be implemented in hardware, software or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation. Any number of the described blocks can be combined in any order and/or in parallel to implement the process, or alternative processes, and not all of the blocks need be executed. For discussion purposes, the processes are described with reference to the environments, systems and devices described in the examples herein, although the processes may be implemented in a wide variety of other environments, systems and devices.

FIG. 7 is a flow diagram illustrating an example process 700 for providing interactive real-time instruction to a user for performing a spot measurement according to some implementations. In some examples, the process 700 may be executed by the user device 104, as discussed above with respect to FIG. 1. For example, the user device 104 may execute the user application, which may utilize an instructional computational model for assessing the user's body position, wearable device position, and so forth, for ensuring consistency between spot measurements for a bodily condition.

At 702, the user device 104 may receive an indication that the user wants to take a spot measurement of a bodily condition. In this example, suppose that the user has selected a user interface virtual control to take a spot measurement of the user's blood pressure, with it being understood that spot measurements of other bodily conditions may be similarly guided by the user application.

At 704, in some examples, the user device 104 may instruct the user to activate the camera on the user device and/or direct the camera toward the user. For example, the camera may be set to a wide-angle setting when available. In some examples, a camera included in smart glasses may be employed with a mirror.

At 706, the user device 104 may receive images from the camera and present the received images of the user in the user interface.

At 708, the user device 104 may use the instructional computational model to determine whether the captured position of the user's body and position of the wearable device are within a threshold range of optimal positions.

At 710, the user device 104 may determine whether the user's body position is within a threshold of the optimal position. In some examples, the instructional computational model may employ one or more images from past spot measurements of the user for measuring the same body condition when determining whether the user body is within the threshold of the optimal body position. For instance, as mentioned above, the optimal body position may be dependent in part on the type of bodily condition being measured. For example, measuring body temperature may not require any particular body position, whereas for measuring blood pressure or generating an ECG, the optimal body position may be sitting, reclining, or the like. Further, the threshold may also be dependent on the type of bodily condition being measured, and may be determined at least in part based on how much a difference in body position might affect the accuracy of the measurement of the bodily condition. If the user's body is within a threshold range of the optimal body position, the process goes to 714. If not, the process goes to 712 to instruct the user to reposition the user's body.

At 712, when the instructional computational model determines that the users body position is not within a threshold range of an acceptable position, the user device 104 may instruct the user to reposition the users body to conform more closely to the optimal body position. In some cases, as discussed above, the instructions may include text and spoken instructions, and may also include graphic overlays and/or animations or images overlaid on, or interleaved with, in real time, the live image of the user presented in the user interface.

At 714, in some examples, when the user's body position is determined to be within a threshold of the optimal position, the user device 104 may determine whether the position of the wearable device with respect to the user's body is within a threshold of the optimal wearable device position. Similar to the threshold for the optimal body position, the threshold for the optimal wearable device position may be dependent in part on the type of bodily condition being measured. For example, measuring body temperature may not require any particular wearable device position, whereas for measuring blood pressure or generating an ECG, the optimal wearable device position may include having the user's hand with the wearable device resting on a surface or the like. Further, the quantification of the threshold may also be dependent on the type of bodily condition being measured, and may be determined at least in part based on how much a difference in the position of the wearable device might affect the accuracy of the measurement of the bodily condition. If the position of the wearable device with respect to the user's body is within the threshold of the optimal wearable device position, the process goes to 718. If not, the process goes to 716.

At 716, if the position of the wearable device 102 is not within the threshold of the optimal position for the wearable device, the user device 104 may provide instructions to the user to reposition the wearable device. For instance, in the case that the spot measurement is blood pressure, the instructions may instruct the user to place the user's hand on which the wearable device is mounted on a surface close to the level of the user's heart and with the palm facing up. In some cases, as discussed above, the instructions may include text and spoken instructions, and may also include graphic overlays and/or animations or images overlaid on, or interleaved with, in real time, the live image of the user presented in the user interface.

At 718, when the body position and wearable device position are both within their respective thresholds of their respective optimal positions, the user device 104 may provide additional instructions to the user for performing the measurement. Examples may include instructions for breathing, remaining still, and so forth, while the bodily condition is measured.

At 720, the user device 104 may present a countdown timer and may measure the user's bodily condition as the timer counts down. In some examples, the user device 104 may receive sensor data for the spot measurement from the wearable device 102, and may determine the spot measurement result on the user device 104, such as by using one or more bodily condition computational models for determining the spot measurement result from the received sensor data. In other examples, the wearable device 102 may include the one or more bodily condition computational models and may determine the spot measurement result on the wearable device. In this configuration, the wearable device 102 may send the spot measurement result to the user device 104 with or without the corresponding sensor data.

At 722, the user device 104 may present and/or store the measured bodily condition value. In some cases, the user device 104 may also send the result to the wearable device 102, such as in the case that the spot measurement result was determined on the user device 104 and the wearable device 102 has a display 110 for presenting the spot measurement result.

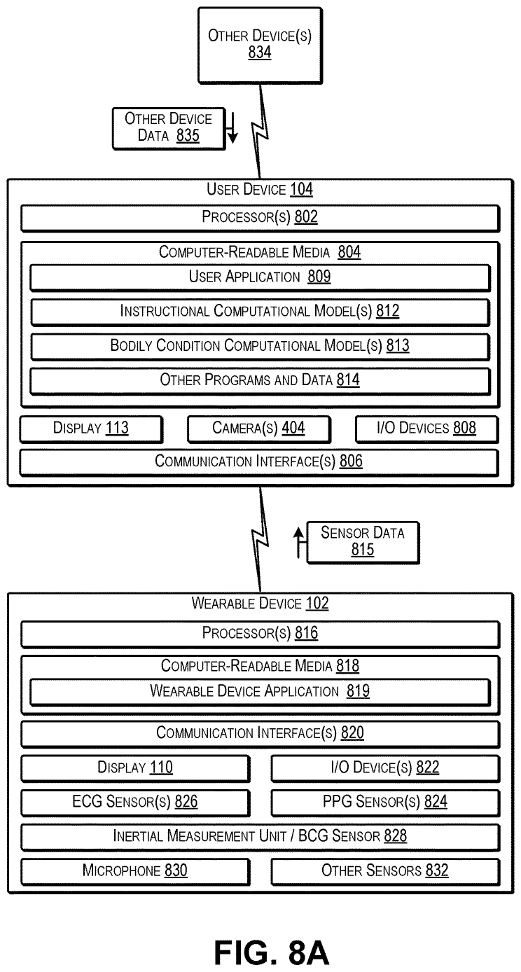

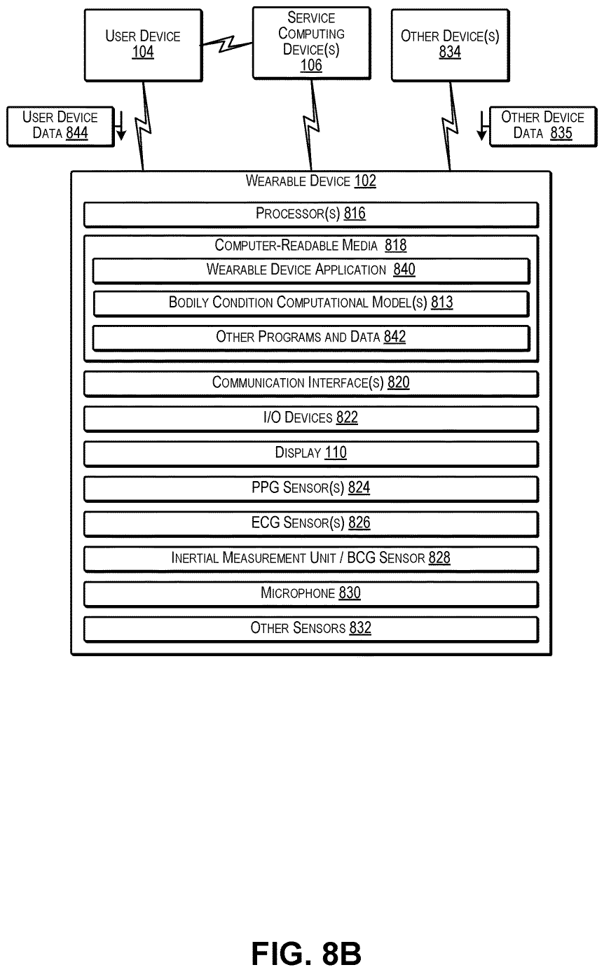

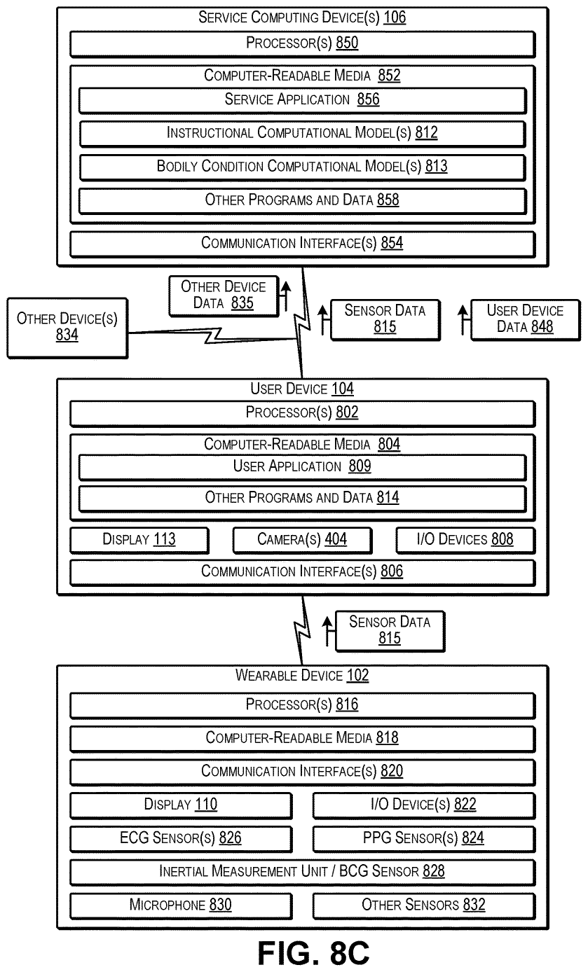

FIGS. 8A-8C illustrate several examples of various system configurations able to perform at least some of the functionality described herein. FIG. 8A illustrates select example components of the user device 104 and the wearable device 102 that may be used to implement at least some of the functionality herein according to some implementations. The user device 104 may include any of a number of different types of computing devices. Some examples of the user device 104 may include smartphones and mobile communication devices; tablet computing devices; laptops, netbooks, and other mobile computers; desktop computers and terminals; smart glasses, augmented reality glasses, augmented reality headsets; and any other device capable of sending and receiving communications and performing the functions according to the techniques described herein.

In the example of FIG. 8A, the user device 104 includes components such as at least one processor 802, one or more computer-readable media 804, one or more communication interfaces 806, and one or more input/output (I/O) devices 808. Each processor 802 may itself comprise one or more processors or processing cores. For example, the processor 802 can be implemented as one or more microprocessors, microcomputers, microcontrollers, digital signal processors, central processing units, artificial intelligence processing units, system-on-chips, graphics processing units, state machines, logic circuitries, and/or any devices that manipulate signals based on operational instructions. In some cases, the processor(s) 802 may be one or more hardware processors and/or logic circuits of any suitable type specifically programmed or configured to execute the algorithms and processes described herein. The processor(s) 802 can be configured to fetch and execute computer-readable processor-executable instructions stored in the computer-readable media 804.

Depending on the configuration of the user device 104, the computer-readable media 804 may be an example of tangible non-transitory computer storage media and may include volatile and nonvolatile memory and/or removable and non-removable media implemented in any type of technology for storage of information such as computer-readable, processor-executable instructions, data structures, program modules, or other data, executable code or the like. The computer-readable media 804 may include, but is not limited to, RAM, ROM, EEPROM, flash memory, solid-state storage, magnetic storage, optical storage, and/or other computer-readable media technology. Further, in some cases, the user device 104 may access external storage, such as storage arrays, network-attached storage, storage area networks, cloud storage, or any other medium that can be used to store information and that can be accessed by the processor(s) 802 directly or through another computing device or network. Accordingly, the computer-readable media 804 may be computer storage media able to store instructions, programs, or components that may be executed by the processor(s) 802. Further, when mentioned herein, non-transitory computer-readable media exclude media such as energy, carrier signals, electromagnetic waves, and signals per se.

The computer-readable media 804 may be used to store and maintain any number of functional components that are executable by the processor(s) 802. In some implementations, these functional components comprise instructions or programs that are executable by the processor(s) 802 and that, when executed, implement operational logic for performing the actions and services attributed above to the user device 104. Functional components of the user device 104 stored in the computer-readable media 804 may include a user application 809 that enables the processor(s) 802 to receive sensor data from the wearable device 102 and determine values of one or more bodily conditions from the sensor data. The user application 809 may present the determined bodily condition values on a display 810 associated with the user device 104, and/or may communicate the bodily condition values to another computing device and/or the wearable device 102, and/or may communicate the sensor data to another computing device, such as the network-located service computing devices discussed above with respect to FIG. 1, which, in some examples, may calculate the bodily condition values instead of the user device 104. Additional description of determining bodily condition values is described in U.S. patent application Ser. No. 18/937,625, filed on Nov. 5, 2024, which has been incorporated by reference herein above. In addition, the user application 809 may provide interactive real-time instructions to a user, such as for providing instructions to the user for taking spot measurements of the various bodily conditions.

In addition, the computer-readable media 804 may also store data, data structures, computational models, and the like, that are used by the functional components. Thus, the computer readable media 804 may store one or more instructional computational models 812 as discussed above. For instance, the instructional computational model 812 may be employed by the user application 809 for providing instructions to a user regarding how to take a spot measurement for a particular bodily condition such as blood pressure, EKG, blood oxygen level, and so forth, as listed more fully elsewhere in this disclosure.

Furthermore the user application 809 may use one or more bodily condition computational models 813 for determining measured values of the various bodily conditions based on sensor data received from the wearable device 102. As one example, a single bodily condition computational model 813 may be employed as a joint model that is trained for receiving multiple different types of sensor data as inputs for determining one or more bodily conditions. In other examples, a plurality of bodily condition computational models 813 may be employed by the user application 809, each of which may be trained to receive a respective different type of sensor data for determining one or more bodily conditions. Details of the bodily condition computational model(s) 813 are disclosed in U.S. patent application Ser. No. 18/937,625, filed on Nov. 5, 2024, which has been incorporated by reference above.

Depending on the type of the user device 104, the computer-readable media 804 may also optionally include other functional components and data, such as other programs and data 814, which may include applications, programs, drivers, etc., and the data used or generated by the functional components, computational models, and the like. Further, the user device 104 may include many other logical, programmatic, and physical components, of which those described are merely examples that are related to the discussion herein.

The communication interface(s) 806 may include one or more interfaces and hardware components for enabling communication with various other devices, such as over the network(s) 108 or directly. For example, communication interface(s) 806 may enable communication through one or more of the Internet, cable networks, cellular networks, wireless networks (e.g., Wi-Fi) and wired networks (e.g., fiber optic, Ethernet), as well as close-range communications such as BLUETOOTH®, BLUETOOTH® low energy, and the like. Implementations herein are not limited to any particular communication protocol or network type.

The user device 104 may further include the one or more I/O devices 808. The I/O devices 808 may include speakers, a microphone, and various user controls (e.g., a touchscreen, buttons, a joystick, a keyboard, a keypad, etc.), a haptic output device, and so forth. Other components included in the user device 104 may include various types of sensors (not shown), which may include a satellite positioning system receiver, an accelerometer, gyroscope, compass, IMU, proximity sensor, and the like.

FIG. 8A further illustrates that the user device 104 may include the display 113 discussed above with respect to FIG. 1, and which may be any suitable type of electronic display device for visually presenting information, user interfaces, or the like, and which may include an associated touchscreen for receiving user inputs. In addition, the user device 104 may include the one or more cameras 404 that enable the user to take an image, video, or the like, such as to provide to the user application 809, and to enable the user application 809 to provide interactive real-time instructions to the user for instructing the user on optimal positioning for taking a spot measurement.

The user device 104 may be able to communicate with the wearable device 102, as discussed above with respect to FIG. 1. In this example, the user device 104 may be able to receive sensor data 815 from the wearable device 102 through wired or wireless communication with the wearable device 102. As discussed above, the user application 809 may use one or more bodily condition computational models 813 for calculating one or more bodily condition values based on the received sensor data 815. Alternatively, in other examples, such as discussed below with respect to FIG. 8B, the wearable device 102, may include sufficient processing capability for determining one or more of the bodily condition values onboard the wearable device 102.

In this example, the wearable device 102 includes one or more processors 816, one or more computer readable media 818, one or more communication interfaces 820, and one or more I/O devices 822. For instance, the processors 816 may correspond to any of the processors 802 discussed above with respect to the user device 104. Similarly, the computer readable media 818 may correspond to any of the computer readable media 804 discussed above, the communication interfaces 820 may correspond to any of the communication interfaces 806 discussed above, and the I/O devices 822 may correspond to any of the I/O devices 808 discussed above. The processor(s) 816 may execute a wearable device application 819 that is maintained in then computer-readable media 818, and that may provide functionality to the wearable device 102 for the functions described herein.

The wearable device 102 in this example includes the display 110, which in some examples may be a low-energy-consuming, highly durable display such as an electronic paper display, e.g., an electrophoretic display, an electrowetting display, an organic transistor display, or the like. Alternatively, in other examples, the display 110 may be any suitable type of display technology such as LCD, OLED, or the like. Additionally, the wearable device 102 includes one or more sensors for sensing bodily signals indicative of a bodily condition such as PPG sensor(s) 824, ECG sensor(s) 826, IMU and/or BCG sensor(s) 828, a microphone 830, and/or other sensors 832, such as a proximity sensor, light sensor, and so forth.

Additionally, in some examples, the user device 104 may be able to communication with one or more other devices 834 that may provide other device data 835 to the user device 104. Examples of other device(s) 834 may include a personal computer (PC) (e.g., laptop, desktop, tablet, etc.) smart glasses, a smart watch, or other wearable that the user may be wearing in addition to the wearable device 102, as well as any of numerous other devices that may be in the environment of the user, such as internet-of-things devices, cameras, thermostats, smart speakers, or any other device able to communicate with the user device 104 and able to provide information that may be relevant to the bodily condition being measured, such as the blood pressure, heart rate, etc., of the user.

As one example, the user device 104 may receive, from the one or more other devices 834 in communication with the user device 104, environment information, activity information, and various other types of information related to a current context of the user and/or various considerations that may affect the accuracy of the measurement. For example, the other device information 835 may include information about one or more context factors that may affect the blood pressure of the user, such as by increasing or decreasing the blood pressure of the user as compared to the average blood pressure measurement of the user. As one example, the user device 104 may receive a communication from smart glasses or other wearable devices of the user regarding various activities of the user or other recent events that may have affected the blood pressure of the user. Examples of such factors which may include consumption of caffeinated drinks, consumption of food, consumption of alcohol, performing exercise, smoking, or the like.

As one example, an AI program may be active on the smart glasses or other wearable device of the user, such as by receiving and analyzing a video feed from one or more cameras and microphones of the smart glasses, and the AI program may recognize any food or drink that is consumed by the user as well as the time of the consumption. In addition, the AI program may detect exercise by the user, other activities of the user, such as smoking, vaping, and so forth, and may provide this information to the user application 809 on the user device 104, such as via one or more APIs that enable communication between the user application 809 and the AI program or other application(s) executing in association with the smart glasses. In some examples, an AI program associated with the smart glasses may be configured to maintain detailed records of the food and drink consumed by the user, such as for diet-tracking purposes or the like. As discussed additionally below, the user application 809 and/or the bodily condition computational model 813 may use this information, as well as information that might be provided by the user device 104 itself, for providing certain insights to bodily condition measurements, such as blood pressure measurements, heart rate measurements. For instance, the information may be used for determining a confidence score related to a bodily condition measurement, determining a possible error range for a measurement, determining whether there may be one or more context factors contributing to a particular measurement, such as an elevated blood pressure measurement, and so forth.

FIG. 8B illustrates select example components of a wearable device 102 that may implement at least some of the functionality described herein according to some implementations. In this example, the wearable device 102 is able to perform at least some of the processing for determining the one or more bodily conditions from the sensor data onboard the wearable device 102. As mentioned above, the wearable device 102 may include any of a number of different types of wearable computing devices and/or body-mounted computing devices, which may include watches, bracelets, wristbands, armbands, gloves, belts, chest straps, anklets, rings, goggles, glasses, earphones, helmets, hats, clothing, artificial fingernails, any of the other example wearable devices discussed herein, and any other body-mounted device capable of sending and receiving communications and performing the functions according to the techniques and devices described elsewhere herein.

In the example of FIG. 8B, the wearable device 102 includes the components discussed above, including the one or more processors 816, the one or more computer-readable media 818, the one or more communication interfaces 820, and the one or more input/output (I/O) devices 822, such as described above with respect to FIG. 8A. In addition, the wearable device 102 may include the display 110, as discussed above with respect to FIGS. 1, 2, and 8A, the PPG sensor(s) 824, ECG sensor(s) 826, IMU and/or BCG sensor(s) 828, the microphone 830, and/or other sensors 832, such as a proximity sensor, light sensor, and so forth. In addition, in some examples, the wearable device 102 may receive the other device data 835 from the other devices 834 and/or user device data 844 from the user device 104. As discussed additionally below, the user device data 844 and the other device data 844 may be used for determining a context of the user during measurement, as well as for determining a confidence in results, a possible error range in the results, and may further be input into one or more of the models for improving the accuracy of a measurement result.

The computer-readable media 818 may be used to store and maintain any number of functional components that are executable by the processor(s) 816. In some implementations, these functional components comprise instructions or programs that are executable by the processor(s) 816 and that, when executed, implement operational logic for performing the actions and services attributed above to the wearable device 102. Functional components of the wearable device 102 stored in the computer-readable media 818 in this example may include a wearable device application 840 that enables the processor(s) 816 to receive sensor data from one or more of the sensors 824-832, and determine one or more bodily conditions based on the received sensor data.

The wearable device application 840 may present the resulting bodily condition measurement value(s) on the display 110 associated with the wearable device 102, and/or may communicate the bodily condition measurement values to the user device 104 and/or the service computing devices 106 discussed above with respect to FIG. 1 such as via close-range radio (e.g., BLUETOOTH®), Wi-Fi, cellular radio, or the like, using an API or the like. In some cases, the wearable device 102 may be configured to communicate primarily with the user device 104, and communicate with the service computing device(s) 106 and/or other devices 834 through the user device 104. In other cases, the wearable device 102 may communicate directly with the service computing device(s) 106 and/or the other devices 834, in addition to the user device 104. Additionally, in some cases, the wearable device 102 may also send the raw sensor data to another computing device, such as the user device 104 and/or the service computing device(s) 106, such as for maintaining a database of the raw sensor data in association with the determined bodily condition measurements derived therefrom.

In addition, the computer-readable media 818 may also store data, data structures, computational models, and the like, that are used by the functional components. Thus, the computer-readable media 818 may store one or more bodily condition computational models 813, as discussed above. For instance, as one example, a single bodily condition computational model 813 may be employed as a joint model trained for receiving multiple different types of sensor data as inputs. In other examples, a plurality of bodily condition computational models 813 may be employed, each of which may be trained to receive respective different sensor data from a different sensor type.

Depending on the type of the wearable device 102, the computer-readable media 818 may also optionally include other functional components and data, such as other programs and data 842, which may include applications, programs, drivers, etc., and the data used or generated by the functional components. Further, the wearable device 102 may include many other logical, programmatic, and physical components, of which those described are merely examples that are related to the discussion herein.

FIG. 8C illustrates select example components of the service computing device(s) 106, the user device 104, and the wearable device 102 that may be used to implement at least some of the functionality herein according to some implementations. Each service computing device 106 may include one or more servers or other types of computing devices that may be embodied in any number of ways. For instance, in the case of a server, the programs, other functional components, and data may be implemented on a single server, a cluster of servers, a server farm or data center, a cloud-hosted computing service, and so forth, although other computer architectures may additionally or alternatively be used. Multiple service computing devices 106 may be located together or separately, and organized, for example, as virtual servers, server banks, and/or server farms. The described functionality may be provided by the servers of a single entity or enterprise, or may be provided by the servers and/or services of multiple different entities or enterprises.

In the illustrated example, the service computing device 106 includes, or may have associated therewith, one or more processors 850 one or more computer-readable media 852, and one or more communication interfaces 854. Each processor 850 may be a single processing unit or a number of processing units, and may include single or multiple computing units, or multiple processing cores. The processor(s) 850 can be implemented as one or more central processing units, microprocessors, microcomputers, microcontrollers, digital signal processors, system-on-chip processors, graphics processing units, artificial intelligence processing units, embedded processors, state machines, logic circuitries, and/or any devices that manipulate signals based on operational instructions. As one example, the processor(s) 850 may include one or more hardware processors and/or logic circuits of any suitable type specifically programmed or configured to execute the algorithms and processes described herein. The processor(s) 850 may be configured to fetch and execute computer-readable instructions stored in the computer-readable media 852, which may program the processor(s) 850 to perform the functions described herein.

The computer-readable media 852 may include volatile and nonvolatile memory and/or removable and non-removable media implemented in any type of technology for storage of information, such as computer-readable instructions, data structures, program modules, or other data. For example, the computer-readable media 852 may include, but is not limited to, RAM, ROM, EEPROM, flash memory or other memory technology, optical storage, solid state storage, magnetic tape, magnetic disk storage, storage arrays, directly attached storage, network attached storage, storage area networks, cloud storage, combinations thereof, or any other medium that can be used to store the desired information and that can be accessed by a computing device. Depending on the configuration of the service computing device(s) 106, the computer-readable media 852 may be a tangible non-transitory medium to the extent that, when mentioned, non-transitory computer-readable media exclude media such as energy, carrier signals, electromagnetic waves, and/or signals per se. In some cases, the computer-readable media 852 may be at the same location as the service computing device 106, while in other examples, the computer-readable media 852 may be partially remote from the service computing device 106.

The computer-readable media 852 may be used to store any number of functional components that are executable by the processor(s) 850. In many implementations, these functional components comprise instructions or programs that are executable by the processor(s) 850 and that, when executed, specifically program the processor(s) 850 to perform the actions attributed herein to the service computing device 106. Functional components stored in the computer-readable media 852 may include a service application 856, which may include one or more computer programs, applications, executable code, or portions thereof. For example, the service application 856 may be executed to receive data for determining a bodily condition of a user, may input at least some of the received data into one or more bodily condition computational models 813, and may return a result including a measurement of the bodily condition, as well as context information, confidence scores, possible error ranges, and so forth.

In addition, the computer-readable media 852 may store data, data structures, and other information used for performing the functions and services described herein. For example, the computer-readable media 852 may store the computational models 812 and 813, as other programs and data 858. For example, among other things, the other programs and data 858 may include programs and training data for training and testing the computational models 812 and 813.

Further, while these data structures and programs are illustrated together in this example, during use, some or all of these data structures and/or programs may be stored on separate service computing device(s) 106. The service computing device 106 may also include or maintain other functional components and data, which may include programs, drivers, etc., and the data used or generated by the functional components. Further, the individual service computing devices 106 may include many other logical, programmatic, and physical components, of which those described above are merely examples that are related to the discussion herein.

The one or more communication interfaces 854 may include one or more software and/or hardware components for enabling communication with various other devices, such as over the one or more network(s) 108 discussed above. For example, the communication interface(s) 854 may enable communication through one or more of a LAN, the Internet, cable networks, cellular networks, wireless networks (e.g., Wi-Fi) and wired networks (e.g., Fibre Channel, fiber optic, Ethernet), direct connections, as well as close-range communications such as BLUETOOTH®, and the like, as additionally enumerated elsewhere herein.

In the example of FIG. 8C, one or more of the computational models 812, 813 may be executed on one or more of the service computing devices 106. In this case, the user device 104 and/or the wearable device 102 may send the sensor data 815 obtained by the wearable device 102 to the service computing device(s) 106. In addition, the user device 104 may send user device data 848 obtained by the user device 104 to the service computing device 106, such as environment information, user information, context information, information received from applications executing on the user device 104, information from sensor onboard the user device, and so forth. Furthermore, the other device data 835 may be sent to the service computing device(s) 106 directly from the other devices 834, or may be sent by the user device 104 after being received by the user device 104 from the other devices 834.

The service computing device 106 may input the received sensor data 815, user device data 848, another device data 835 into the one or more computational models 813 for determining one or more bodily conditions of the user, such as blood pressure, heart rate, body temperature, respiratory rate, blood oxygen saturation, heart rate variability, blood sugar, and various other physiological characteristics. The service computing device 106 may further determine a confidence score and/or error range associated with each measurement, and may indicate any context factors that may have influenced a measured bodily condition. The network computing device may send the measurement information, confidence scores, error ranges, context factors, and so forth to the user device 104 and/or the wearable device 102 for storage and/or presentation by at least one of the user device 104 or the wearable device 102.

Similarly, in some examples, the instructional computational model(s) 812 may be executed on the service computing device(s) 106 for providing real-time instructions to the user, such as during a spot blood pressure measurement or the like. Furthermore, while several example hardware and logical configurations have been discussed herein with respect to FIGS. 8A-8C, numerous other variations will be apparent to those of skill in the art having the benefit of the disclosure herein.

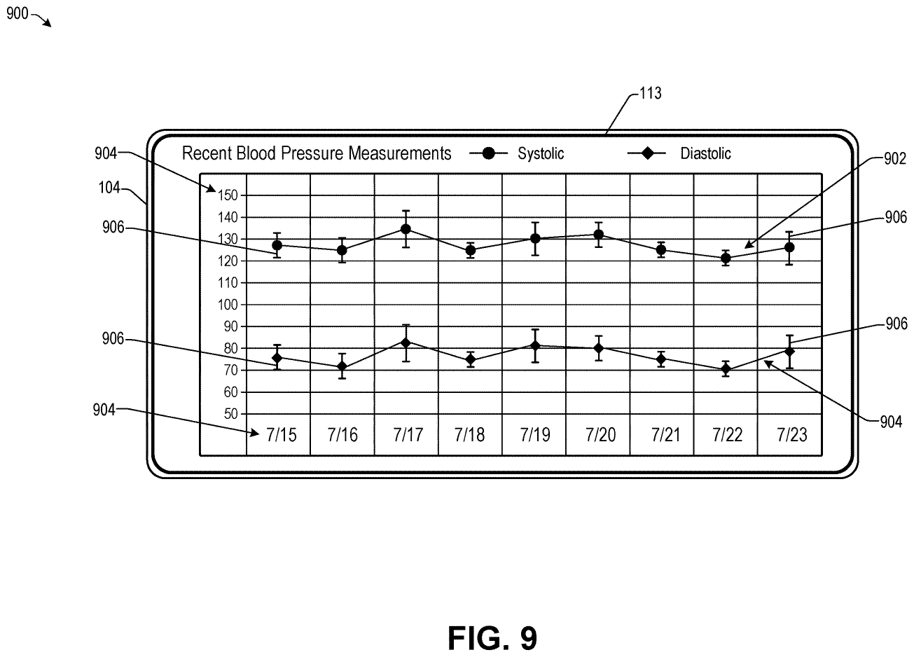

FIG. 9 illustrates an example user interface 900 for presenting a plurality of blood pressure measurements over time with estimated error ranges according to some implementations. In this example, the bodily condition computational model 813 may be configured to output at least one of a confidence score associated with each measurement or an estimated error range associated with each measurement. In some examples, the error range may be determined based on detection of the presence of certain factors that can impact blood pressure by a range that has been studied. For instance, suppose that if the user is talking or moving during the measurement, this has been shown to cause an increase in measured blood pressure by 3-5 points over what would otherwise be the actual reading. Thus the range of possible error in the measurement presented to the user may be based in part on this empirical evidence when such a factor is detected (e.g., by a microphone on the user device or the wearable device). In other examples, the confidence score may be translated to an estimated error range. For example, a measurement with a high confidence score may correspond to a lower range of estimated possible error, while a measurement with a lower confidence score may correspond to a larger range of estimated possible error.

In the illustrated example, the user interface 900 includes a plot 902 of a plurality of systolic blood pressure measurements over time and a plot 904 of a plurality of corresponding diastolic blood pressure measurements over the same time periods. Each systolic and diastolic measurement has an estimated range of possible error in the measurement, as indicated by an error bar 906 shown with each measurement. For instance, each error bar 906 may show a range of possible error within which the actual measurement is estimated to exist.

Further, while error bars are used in this example as one technique for communicating a possible error range, in other examples, other techniques may be used, such as by providing the error range in digits adjacent to the measurement. For example the blood pressure measurement may be displayed as 128±4 over 71±3. Further, while several examples of providing an estimated range of a possible error in a measurement have been described herein, numerous variations will be apparent to those of skill in the art having the benefit of the disclosure herein.

As discussed additionally below, in some implementations, a single computational model may be trained as a joint model that is able to receive inputs of sensor data from multiple different sensor types, such as PPG, ECG, BCG, sound, IMU, as well as numerous other types of sensor data from various other electronic devices and/or other sources of information. For instance, the joint model may incorporate features and/or parameters for all of the different sensor types from which sensor data may be received, as well as other sources of information that may be useful for determining a current condition of a user. The joint computational model may be optimized according to an estimation template that may be applied in one step on all of the inputted sensor data and information for determining an estimated blood pressure measurement of the user and a confidence score indicative of a predicted accuracy of the blood pressure measurement.

As another example, multiple computational models, such as multiple machine learning models, may be trained, with each respective computational model being trained or dedicated for a particular sensor type or other type of information. Under this technique, the system may include an optimized PPG model, an optimized ECG model, an optimized BCG model, and, in some examples, other additional optimized models dedicated for other types of sensor data or other types of received information. The outputs of the multiple computational models may be aggregated, such as based on a bias versus estimator variance, for providing an estimated blood pressure measurement of the user. Furthermore, the aggregated blood pressure measurement may be input to a joint model that is trained to receive inputs for other types of the sensor data or other information for outputting the estimated blood pressure measurement and an associated confidence score.

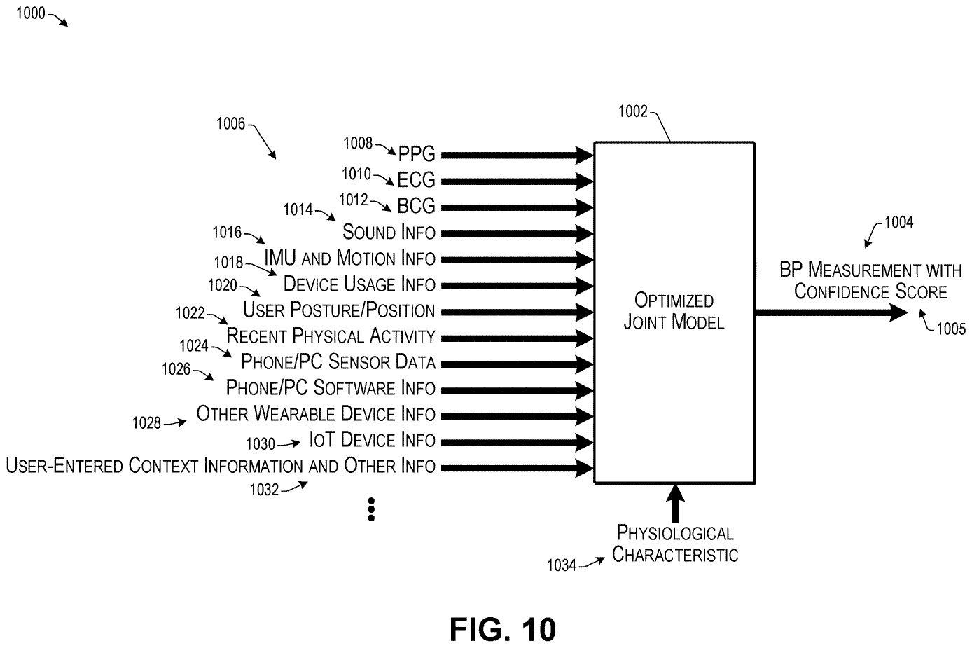

FIG. 10 illustrates an example 1000 of using a computational model configured as an optimized joint model 1002 for determining a blood pressure measurement and confidence score 1104 according to some implementations. For example, such as based on receiving sensor data and other information 1006 as inputs, the optimized joint model 1002 may output an estimated blood pressure measurement 1004 of a user, and may also provide a confidence score 1005 for the blood pressure measurement 1004. For instance, the optimized joint model 1002 may receive PPG sensor data 1008, ECG sensor data 1010, and/or BCG sensor data 1012 that may be used, at least in part, for determining the estimated blood pressure measurement 1004 of the user.

In addition to the sensor data 1008, 1010, and/or 1012, the inputs to the optimized joint model 1002 may include a variety of other sensor data and other information 1006 that may be available from the environment surrounding the user, as well as information received from electronic devices that may be proximate to the user or otherwise available for providing information related to the user and/or activity of the user, and that may be relevant to the blood pressure measurement. These other types of the sensor data and other information 1006 may help to improve the accuracy of the blood pressure measurement 1004 and/or may be used to determine the confidence score 1005 that indicates a predicted accuracy of the blood pressure measurement 1004 that is output by the optimized joint model 1002. For instance the sensor data and other information 1006 may provide information related to current or recent activity of the user, current conditions in the user's environment, a current posture and/or body position of the user, current vital signs of the user (e.g., heart rate, respiration rate, movement of the user, etc.), and so forth. Additionally, the sensor data and other data may provide context information related to one or more context factors that are known to influence the blood pressure of a person, such as exercise, caffeine, eating, smoking, and so forth, as described in additional detail below.

As one example, the sensor data and other information 1006 that may be input into the optimized joint model 1002 may further include sound information 1014 detected in the environment of the user by one or more electronic devices proximate to the user. For example, the sound information 1014 may be detected by the microphone on the user device 104, the wearable device 102, or other devices 834, and may indicate a current activity or condition of the user, or other information about the environment of the user, as well as indicating any sensed bodily conditions of the user that may be detected using sound sensing. As one example, taking a blood pressure measurement in a loud environment with a lot of background noise (or while the user is talking, being talked to, or the like) may cause the user's blood pressure to be elevated and/or can cause the blood pressure measurement itself to be inaccurate. Further, the sound information may also indicate past activity of the user, such as for providing information relevant to one or more context factors as discussed additionally below.

Additionally, in some examples, the BCG sensor may be an accelerometer. In other examples, an IMU may also serve as the BCG sensor while also having the ability to detect other physiological functions such as breathing, physical activity, physical positions, or other detectable information of the user, motion or activity of the user, and the like. Thus, IMU and motion information 1016 may include information from or more of IMUs, accelerometers, gyroscopes, motion sensors, or the like, received from one or more electronic devices worn by the user, mounted on the user, or disposed proximate to the user. The IMU and motion information 1016 may be input into the optimized joint model 1002 to provide additional information for increasing the accuracy of the blood pressure measurement 1004 and/or for providing a confidence score that is associated with the output of the optimized joint model 1002, such as by indicating whether the user is moving or remaining still during the measurement, as well as indicating other conditions or activity of the user that might affect the blood pressure measurement. As one example, the IMU information may be used to detect exercise by the user, and which may be at least temporarily stored with a time at which the exercise was detected, such as for providing context information if a blood pressure measurement is taken within a threshold time of completion of the exercise.

In addition, device usage information 1018 may include information received from one or more electronic devices worn by the user, used by the user, or otherwise proximate to the user, which may include connected devices such as gaming consoles, exercise equipment, thermostats, and any of numerous other connected devices. The device usage information 1018 may provide an indication of how the user is using an electronic device or other connected device, such as in a manner that might affect the accuracy of the blood pressure measurement. For example, the user may be using an application, such as playing a video game, or using an exercise application or apparatus, or the like, that may affect the user's blood pressure. The user application 809 on the user device 104 and/or the wearable device application 840 on the wearable device 102 may be configured to provide the device usage information 1018 to the optimized joint model 1002. Further, the user application 809 on the user device and/or the wearable device application 840 may be configured to communicate with any other electronic devices in the environment of the user that may provide other device usage information 1018 regarding user usage of electronic devices that may affect the blood pressure measurement of the user and/or that may provide context information for one or more context factors that may affect the blood pressure of the user.

As another example, user posture and/or position information 1020 may be received from one or more electronic devices, such as based on video or other image information. For instance a personal computer (PC) (e.g., laptop, desktop, tablet, etc.) smart glasses, phone, or the like, as discussed above, may include a camera for capturing an image of the user, and which may be provided to the optimized joint model 1002 for providing an indication of the user's posture, the position of the user's arms/extremities, and so forth, as discussed above, while taking the blood pressure measurement. As mentioned above, differences in user posture, arm position, and the like, may affect consistency of the blood pressure measurement from one measurement to the next.

As another example, recent physical activity 1022 of the user may be received from a user wearable device, such as the wearable device 102 discussed herein, an activity tracking wearable device, the user device 104, smart glasses 407, or from another electronic device, such as a network-connected treadmill, network-connected bicycle computer, other piece of network-connected exercise equipment, and so forth. The user application 809 on the user device 104 and/or the wearable device application 840 on the wearable device 102 may be configured to provide and/or receive the information regarding any recent physical activity 1022 of the user, and may provide this information as input to the optimized joint model 1022 and/or as context information.

As still another example, user phone and/or PC sensor data 1024 may be received and may be indicative of various conditions or qualities of the user that may affect the blood pressure measurement of the user. For instance, any of the numerous sensors on a mobile device, such as a smartphone, may provide information regarding a condition that may affect the blood pressure measurement of the user, such as the user's location being different from a usual place. For instance, if the user is at a substantially higher altitude than usual, the user's blood pressure may be higher than normal if the user usually takes blood pressure measurements at close to sea level.

As another example, phone and/or PC software information 1026 may provide an indication of current and recent activity of user regarding applications and programs being used or recently used by the user, such as video games, applications that may increase a stress level of a user, or any of various other types of applications or programs. This software usage information may be received from the phone, PC or other electronic devices proximate to the user. As one example, a health or exercise application on the user device 104 may provide information based on user activity, such as exercise, eating, drinking, or the like.

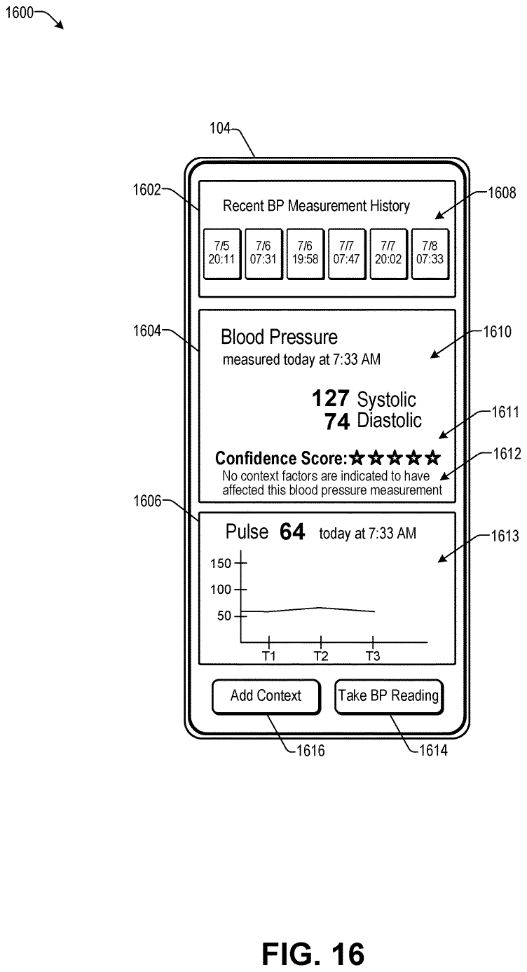

As another example, other wearable device information 1028 may be provided to the optimized joint model. For instance, the user may be wearing a smart watch, a fitness tracker, smart glasses 407, a head-mounted display, a glucose monitor, a pacemaker, defibrillator, or other smart medical device, smart clothing, smart contact lens, smart earbuds, or any of various other connected devices that may be worn and that are able to communicate information about the user to the user device 104 and/or the wearable device 102. As one example, an artificial intelligence (AI) active on smart glasses 407 worn by the user may provide various types of context information to the computational model 1002, and which may be used not only in assisting with the determination of the accuracy of the blood pressure measurement, but which may also be associated with the blood pressure measurement for providing contextual insight related to the blood pressure measurement for various uses, as discussed additionally below, e.g., with respect to FIGS. 16-21.

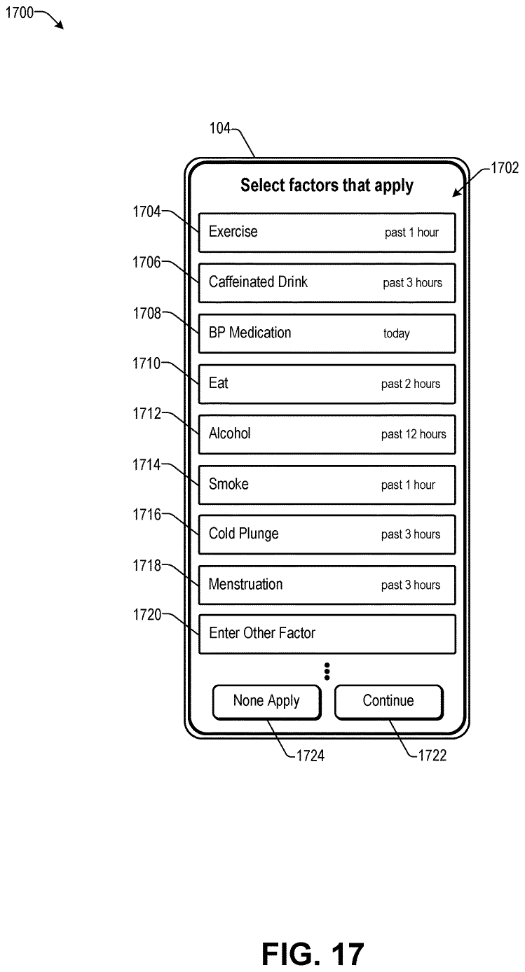

As another example, internet of things (IoT) device information 1030 may be received from connected electronic devices in the environment of the user, such as to indicate a current temperature of the environment of the user, current activities taking place in the environment of the user, and so forth. Additionally, user-entered context information and other information 1032 may include context information entered by the user, as well as any other information that may be received and input into the optimized model that might be relevant to the blood pressure measurement of the user and/or that may provide context relevant to the blood pressure measurement. Thus, examples herein are not limited to the examples of information listed above, but may include additional types of sensor data and/or information that may be relevant to the bodily condition measurement being taken. Further, as discussed additionally below with respect to FIG. 17, a user interface may be presented to the user to enable the user to manually enter context information for events or conditions that may affect the user's blood pressure at the time of a particular blood pressure measurement.

Furthermore, in some examples, at least one physiological characteristic 1034 of a portion of a vascular system of the user corresponding to the received sensor data may be determined and provided to the optimized joint model 1002. For instance, the optimized joint model 1002 may account for arterial and venous characteristics of the user, such as resistance, compliance, and inductance, which may be based in part on physiology for fluid flow and pressure. On the other hand, many conventional systems ignore the differences in the vasculature conditions of individuals, which may vary significantly from one person to the next. Thus, the optimized joint model 1002 herein may take into consideration the individual characteristics of the user's vascular system when determining a blood pressure measurement, thereby improving the accuracy of the measurement. In some examples, the at least one physiological characteristic 1034 may be determined using any of the techniques described in U.S. patent application Ser. No. 18/937,625, filed on Nov. 5, 2024, and which has been incorporated by reference herein.

The optimized joint model 1002 may be optimized using various techniques discussed above. As one example, the optimizing of the joint model 1002 may include comparing an estimated blood pressure output by the model with blood pressure measured using any of the conventional “gold standard” techniques, which may include the use of an inflatable cuff in some examples. For example, during training of the optimized joint model 1002, the difference between the estimated arterial blood pressure determined by the model and the measured arterial blood pressure measured using, e.g., an inflatable cuff device, may be used for adjusting one or more parameters of the estimation model.

Examples of machine-learning models that may be used as the optimized joint model 1002 and the other trainable models described herein may include neural networks such as deep learning models, recurrent neural networks, LSTM models, convolutional neural networks, feedforward neural networks, and so forth, to name a few. Further, other types of machine learning and statistical models may be used, such as models trained using supervised learning algorithms (e.g., Bayesian statistics, support vector machines, decision trees, classifiers, k-nearest neighbor, etc.), unsupervised learning algorithms (e.g., association rule learning, hierarchical clustering, cluster analysis, etc.), semi-supervised learning algorithms, and so forth. Implementations herein are not limited to any particular type of machine learning or otherwise trainable model. Additionally, or alternatively, in some examples, the computational models may include one or more heuristic models and/or mathematical models, or the like, and which may be used to perform some or all of the functions described herein in place of one or more machine learning models or in combination with one or more machine learning models.

As one example of how blood pressure measurement accuracy may be improved, the techniques herein may be able to detect white-coat syndrome or similar conditions that may affect the accuracy of the user's blood pressure measurements. For instance, white-coat syndrome (also known as white-coat hypertension) is a condition in which people exhibit a blood pressure level above their normal range in a clinical setting or otherwise when experiencing anxiety in anticipation of having their blood pressure measured, while not experiencing high blood pressure in other settings. For example, implementations herein may take blood pressure measurements of a patient continuously during the day for use as a reference that takes into account ordinary variations in the user's levels of daily stress and anxiety. Additionally, implementations herein further measure the user's heart rate, respiration rate, and the like, and can determine these signals during a calm period that may be instructed before the user takes a spot blood pressure measurement. Based on this information, the system herein may detect white-coat syndrome if the user suffers anxiety during the reading, causing the user's blood pressure to spike and resulting in an inaccurate measurement.

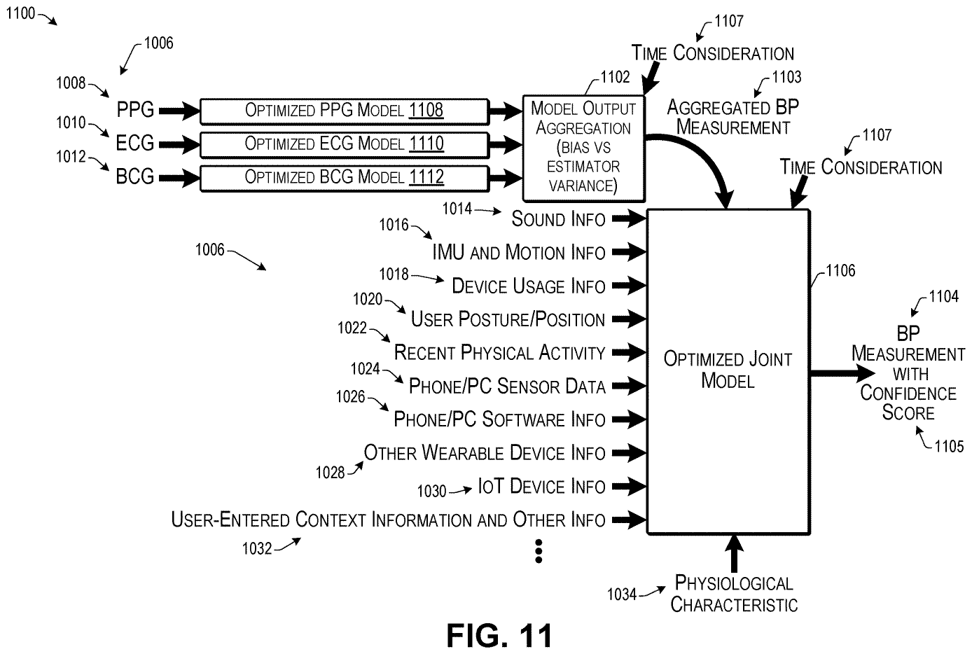

FIG. 11 illustrates an example 1100 of using at least one optimized dedicated model and an optimized joint model for determining an estimated blood pressure measurement 1104 and an associated confidence score 1105 according to some implementations. In this example, each respective optimized dedicated model may be a respective computational model that is trained and provided for each of one or more different types of sensor data, such as the PPG data 1008, the ECG data 1010, and/or the BCG data 1012. Accordingly, an optimized PPG model 1108 may be employed for receiving PPG sensor data 1008 as an input and providing a blood pressure measurement as an output; an optimized ECG model 1110 may be employed for receiving ECG data 1010 as an input and providing a blood pressure measurement as an output; and/or an optimized BCG model 1112 may be employed for receiving BCG data 1012 as an input and providing a blood pressure measurement as an output. In this example, each of the models 1108-1112 that are used may output an estimated blood pressure measurement, and the outputs may be aggregated by an aggregating algorithm at 1102 to provide an aggregated blood pressure measurement 1103 that is provided to the optimized joint model 1106.

As one example, the aggregating algorithm at 1102 may include a biased-versus-estimator variance that provides weighting to the outputs of certain ones of the dedicated models 1108, 1110, and/or 1112, as compared to the outputs of other ones of the dedicated models 1108, 1110, and/or 1112. As one example, the output of the optimized PPG model 1108 may be given greater weight than the output of the optimized BCG model 1112 or the optimized ECG model 1110, if used. Further, while three sensor types are illustrated with respective dedicated optimized models 1108-1112 in this example, in other examples additional sensor types or fewer sensor types may be provided with dedicated optimized models for aggregation at 1102.

The aggregated blood pressure measurement 1103 may be provided to the optimized joint model 1106 which may be trained to take into consideration any of the numerous other ones of the sensor data and other information 1006 discussed above with respect to FIG. 10. Accordingly, the optimized joint model 1106 may receive the sound information 1014, the IMU and motion information 1016, the device usage information 1018, the posture and/or position information 1020, the recent physical activity information 1022, the phone and/or PC sensor data 1024, the phone/PC software information 1126, the other wearable device information 1028, the IoT device data 1030, and/or the user-entered context information and other information 1032 that may be relevant to determining an accurate blood pressure measurement.

Additionally, in some cases, a time consideration 1107 may be taken into account by the aggregating algorithm at 1102 and/or by the optimized joint model 1106, and which is used for determining how the blood pressure estimate is changing over time for providing a more accurate estimation of the blood pressure. For example, by looking at consecutive aggregated blood pressure measurements 1103, or blood pressure measurement estimates over a small time interval, a confidence metric or stability of estimation can be formed. This may be used to guide the aggregating algorithm 1102 and/or the optimized joint model 1106 to accept or reject an aggregated blood pressure measurement 1103. Additionally, the signal quality of the input measurements may vary over time (e.g., due to poor connection or sensor attachment). Therefore, the aggregating algorithm 1102 and/or the optimized joint model 1106 may use a technique such as SQA (signal quality assessment) for individual estimators/sensors to judiciously combine these results. For example, the optimized joint model 1106 or the aggregating algorithm 1102 may choose to drop a blood pressure estimate during certain periods of receiving a poor signal from one or more of the sensors 1008, 1010, or 1012.

The optimized joint model 1106 receives the aggregated blood pressure measurement 1103 and, in some examples, the time consideration 1107, as well as the sensor data and other information 1014-1032 as inputs. Additionally, in some examples, at least one physiological characteristic 1034 of a portion of a vascular system of the user corresponding to the received sensor data may be determined and provided to the optimized joint model 1106. Alternatively, in other examples, the at least one physiological characteristic 1034 may be provided to the optimized PPG model 1108 and/or the optimized ECG model 1110, if any, and/or the optimized BCG model 1112, if any, instead of, or in addition to being provided to the optimized joint model 1106. Based on these inputs, the optimized joint model 1106 outputs the blood pressure measurement 1104 and the associated confidence score 1105 that provides an indication of a predicted accuracy of the blood pressure measurement 1104. As discussed below, in some examples, the confidence score 1105 may be expressed using a star-rating or other easily understandable graphical representation.

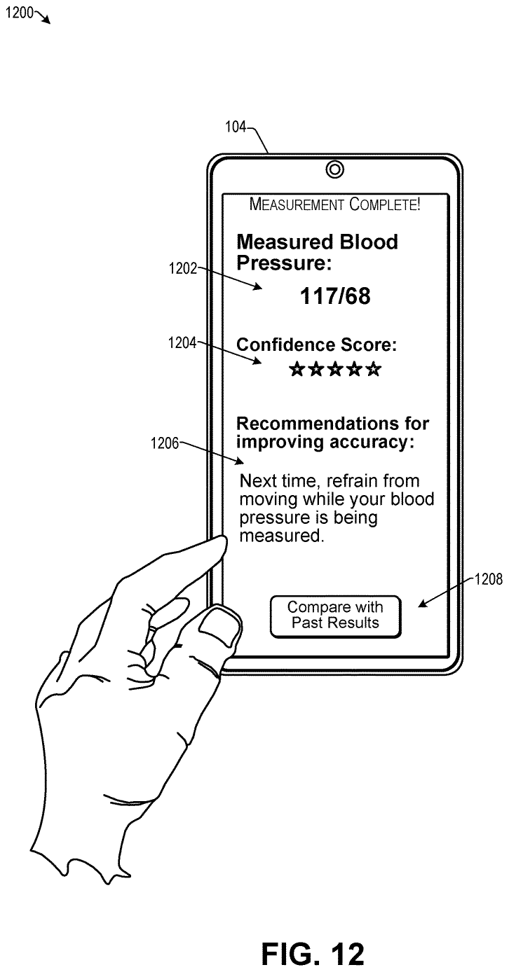

FIG. 12 illustrates an example user interface 1200 that may be presented on the user device 104 for providing blood pressure measurement information to the user after taking a spot blood pressure measurement according to some implementations. The user interface 1200 may be presented by the user application executing on the user device 104 after the user takes a spot blood pressure measurement for measuring the blood pressure of the user. In some examples, the user application may employ the instructional computational model that may check the user's body position (e.g., sitting, standing, etc.) and the location of the wearable device 102 relative to the user's body. Further, the user device 104 may request or otherwise receive any of the additional types of sensor data and other information 1006 discussed above, such as the PPG sensor data 1008, the ECG sensor data 1010, the BCG sensor data 1012, the sound information 1014, the IMU and motion information 1016, the device usage information 1018, the posture and/or position information 1020, the recent physical activity information 1022, the phone and/or PC sensor data 1024, the phone/PC software information 1126, the other wearable device information 1028, the IoT device data 1030, and/or the user-entered context information and other information 1032 that may be relevant to determining an accurate blood pressure measurement.

When the blood pressure measurement is sufficiently completed, the blood pressure measurement and confidence score are output, such as by using one of the computational model configurations discussed above with respect to FIG. 10 or 11. The user interface 1200 presents the measured blood pressure 1202 and the associated confidence score 1204. In the illustrated example, the confidence score is presented as a number of stars, such as between 1 and 5 stars, with a lower number of stars indicating a lower confidence score 1204 and a higher number of stars indicating a higher confidence score, where a higher confidence score indicates a higher likelihood that the measured blood pressure is an accurate measurement. Further, while the confidence score 1204 is presented in this example as a plurality of stars to provide a graphical representation of the confidence score, in other examples, the confidence score may be presented as a numerical value, e.g., a percentage of 100, or using any other type of graphical representation, such as a bar graph, a color coded graphic, combinations of text and graphics, or the like.

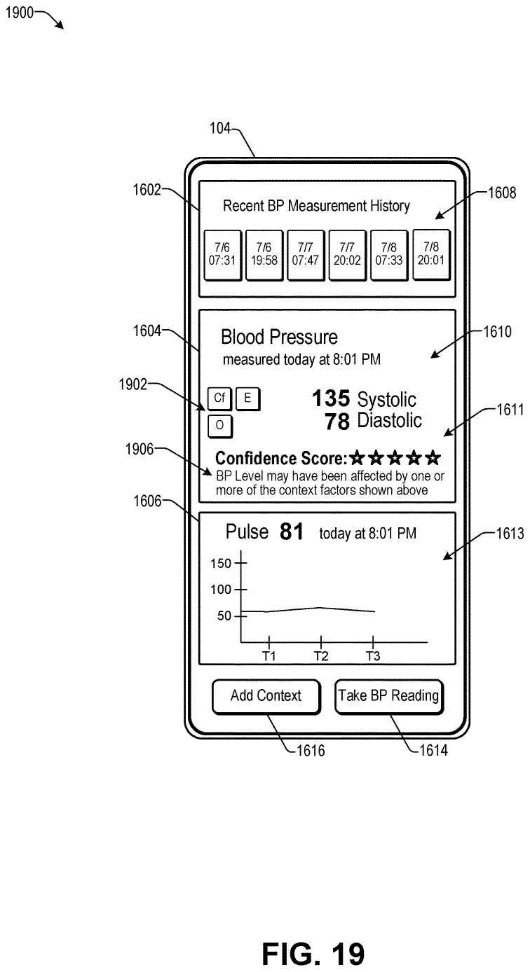

In this example, the user interface may further include one or more recommendations 1206 for improving the accuracy of the blood pressure measurement. For example, if one or more of the inputs to the model, such as the IMU and motion information 1016 and/or the posture and/or position information 1020 indicated that the user was moving while the blood pressure was being measured, the recommendation at 1206 may recommend that the user refrain from moving during future spot testing for blood pressure. Additionally, in this example, the user may select a virtual button 1208 for comparing the currently presented results with past results for that same bodily condition measurement. In some examples, selection of the button 1208 may cause the user application on the user device 104 to present the user interface 1600/1900 discussed below with respect to FIGS. 16 and 19, respectively.

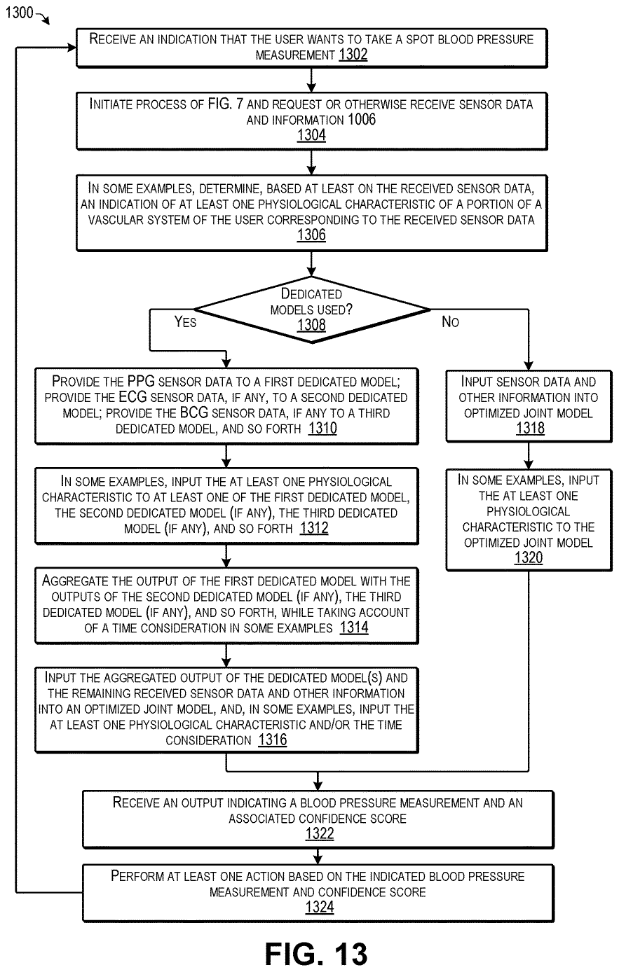

FIG. 13 illustrates an example process 1300 for determining a blood pressure measurement and confidence score according to some implementations. In some examples, the process 1300 may be executed by a suitable computing device, such as by the wearable device 102 and/or the user device 104, as discussed above with respect to FIG. 1, or by any other computing device that may receive the sensor data and information 1006, and that may execute one or more computational models. For example, the computing device may determine the blood pressure measurement at least in part by executing the user application 809 and/or the wearable device application 840.

At 1302, the user device 104 may receive an indication that the user wants to take a spot measurement of a bodily condition. In this example, suppose that the user has selected a user interface virtual control, such as by tapping on a virtual button, to take a spot measurement of the user's blood pressure, with it being understood that spot measurements of other bodily conditions may be similarly guided by the user application.

At 1304, the computing device may initiate the process 700 of FIG. 7 and may request or otherwise receive sensor data and other information 1006 discussed above. Accordingly, the computing device may receive the PPG sensor data 1008, and one or more of the ECG sensor data 1010, the BCG sensor data 1012, the sound information 1014, the IMU and motion information 1016, the device usage information 1018, the posture and/or position information 1020, the recent physical activity information 1022, the phone and/or PC sensor data 1024, the phone/PC software information 1126, the other wearable device information 1028, the IoT device data 1030, and/or the user-entered context information and other information 1032 that may be relevant to determining an accurate blood pressure measurement.

At 1306, in some examples, the computing device may determine, based at least on the received sensor data, an indication of at least one physiological characteristic of a portion of a vascular system of the user corresponding to the received sensor data. Any of the techniques for determining the physiological characteristic described in U.S. patent application Ser. No. 18/937,625, filed on Nov. 5, 2024, which has been incorporated by reference herein, may be used.

At 1308, the computing device may determine whether to use one or more dedicated models for the received sensor data or to use a joint model for all received sensor data and other information. If one or more dedicated models are to be used, the process goes to 1310. Otherwise, the process goes to 1318.

At 1310, the computing device may provide the PPG sensor data to a first dedicated model; provide the ECG sensor data, if any, to a second dedicated model; provide the BCG sensor data, if any, to a third dedicated model, and so forth.

At 1312, in some examples, the computing device may input the at least one physiological characteristic to at least one of the first dedicated model, the second dedicated model (if any), the third dedicated model (if any), and so forth.

At 1314, the computing device may aggregate the output of the first dedicated model with the outputs of the second dedicated model (if any), the third dedicated model (if any), and so forth, while taking account of a time consideration in some examples.

At 1316, the computing device may input the aggregated output of the dedicated model(s) and the remaining received sensor data and information 1006 into an optimized joint model, and, in some examples, input the at least one physiological characteristic and/or the time consideration.

At 1318, when sensor-specific dedicated models are not used, the computing device may provide the received sensor data and information 1006 to an optimized joint computational model that is trained to receive multiple types of sensor data and other information.

At 1320, in some examples, the computing device may provide the at least one physiological characteristic to the optimized joint computational model.

At 1322, the computing device may receive an output of the joint computational model of 1316 or 1320 indicating a blood pressure measurement and an associated confidence score.

At 1324, the computing device may perform at least one action based on the indicated blood pressure measurement and associated confidence score. For example, the computing device may present the blood pressure measurement and associated confidence score on a display associated with the computing device, such as discussed above with respect to FIG. 12. As another example, the computing device may send the blood pressure measurement and associated confidence score to another device to cause the other device to perform an action, such as initiating an alert, presenting the blood pressure measurement and associated confidence score on a display, performing further processing with the blood pressure measurement and associated confidence score, or the like. Further, the process 1300 may return to 1302 to await a next indication from the user.

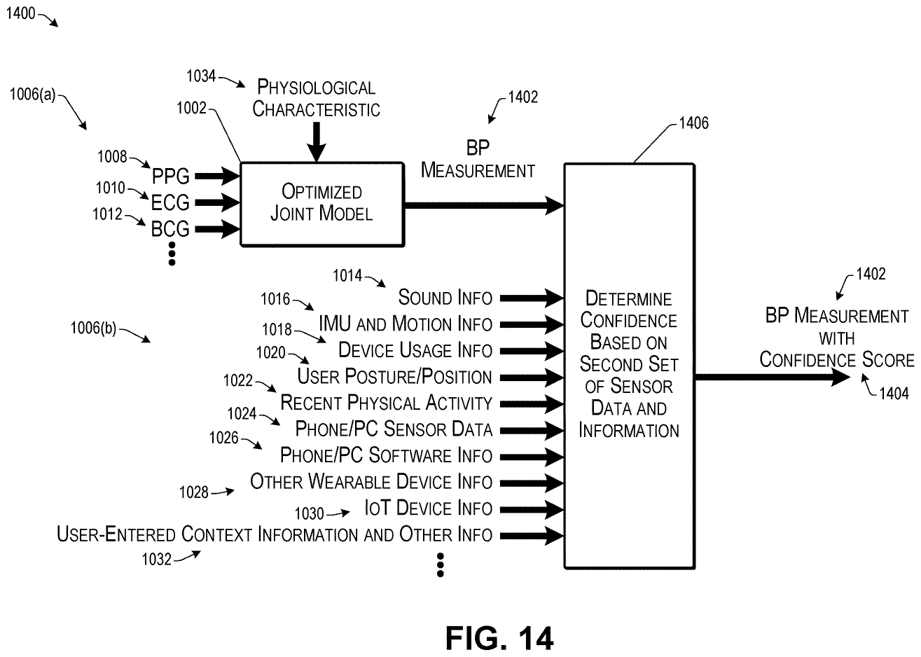

FIG. 14 illustrates an example 1400 of using a computational model configured as an optimized joint model for determining a blood pressure measurement based on a first set of sensor data and/or information, and determining a confidence score based on a second set of sensor data and/or information according to some implementations. For example, a first set of sensor data and/or information 1006(a), such as PPG data 1008, ECG data 1010, and/or BCG data 1012, and so forth, may be input to an optimized joint model 1002, as discussed above, to determine a blood pressure measurement 1402. In some cases, the physiological characteristic 1034 may also be input to the optimized joint model 1002. As indicated at 1406, a second set of at least one of sensor data or other information 1006(b) may then be used to determine a confidence score 1404 by applying one or more algorithms, heuristics, machine learning, or the like, based on the types of additional sensor data or other information 1014-1032 available. For example, if IMU and motion information 1016 or sound information 1014 indicates that the user was moving or performing other activities during the blood pressure measurement, the confidence score 1404 may be lower than if these signals were not detected. Additionally, if one or more context factors are present during the blood pressure measurement, such as recent caffeine consumption, recent exercise, or the like, and the user's blood pressure is somewhat elevated as compared to the user's average blood pressure measurement, then this context information might have no effect on the confidence score, or may have a positive effect on the confidence score (i.e., increased confidence), depending on how the model is trained. Additional details of determining and applying context factors are discussed below, e.g., with respect to FIGS. 16-21.

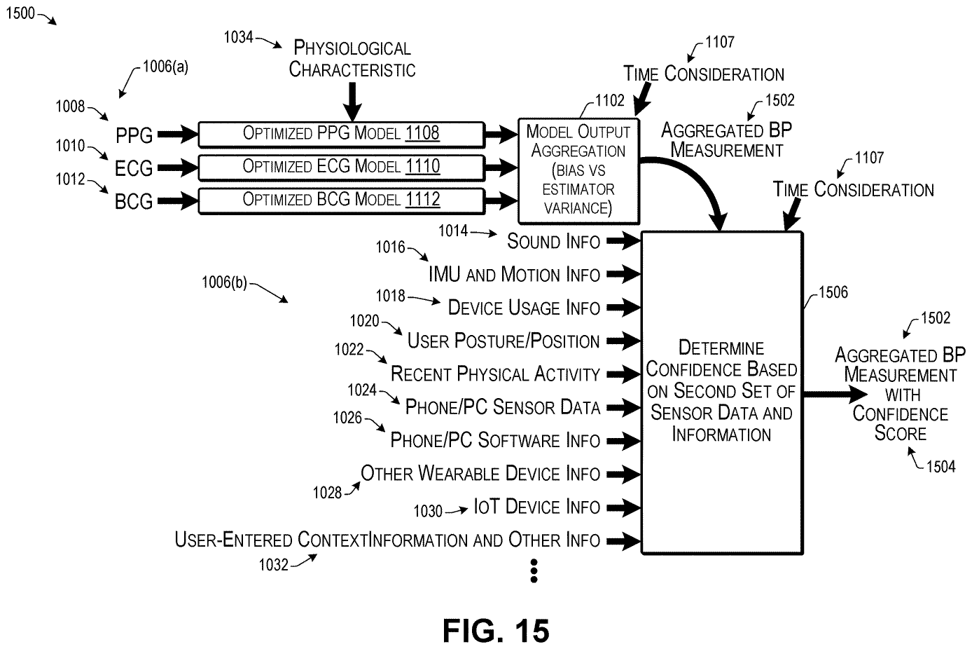

FIG. 15 illustrates an example 1500 of using one or more dedicated optimized models for determining an estimated blood pressure measurement based on a first set of sensor data and/or information, and determining a confidence score based on a second set of sensor data and/or information according to some implementations. For example, a first set of sensor data and/or information 1006(a), such as PPG data 1008, ECG data 1010, and/or BCG data 1012, and so forth, may be input to dedicated optimized computational models, such as an optimized PPG model 1108, an optimized ECG model 1110, and/or an optimized BCG model 1112, respectively, and so forth, as discussed above. The outputs of the one or more dedicated optimized models 1108, 1110, and/or 1112, may be aggregated at 1102, as discussed above, to determine an aggregated blood pressure measurement 1502. In some cases, the physiological characteristic 1034 may also be input to one or more of the dedicated optimized models 1108, 1110, and/or 1112. Additionally, the time consideration 1107 may also be applied in some cases, as discussed above, such as to remove suspect sensor readings.

As indicated at 1506, a second set of at least one of sensor data or other information 1006(b) may then be used to determine a confidence score 1504 for the aggregated blood pressure measurement 1502 by applying one or more algorithms, heuristics, machine learning, or the like, based on the types of additional sensor data or other information 1014-1032 available. For example, if IMU and motion information 1016 or sound information 1014 indicates that the user was moving or performing other activities during the blood pressure measurement, the confidence score 1504 may be lower than if these signals were not detected. On the other hand, if one or more context factors are present during the blood pressure measurement, such as recent caffeine consumption, recent exercise, or the like, and the user's blood pressure is somewhat elevated as compared to the user's average blood pressure measurement, then this context information might have no effect on the confidence score, or may have a positive effect on the confidence score (i.e., increased confidence), depending on how the model is trained. Additional details of determining and applying context factors are discussed below, e.g., with respect to FIGS. 16-21.

Furthermore, attempting to measure the blood pressure of the user while the user is sleeping may pose additional challenges that are not typically encountered while measuring the blood pressure of the user while awake. For example, during sleep the user may be in any of numerous different positions throughout the night, may have the arm with the wearable device and the sensor positioned away from the user's heart, such as over that user's head, pinned underneath the user's body, and so forth, which can lead to large variations in the accuracy of the blood pressure measurement taken while the user is asleep.

Furthermore, some people can have normal blood pressure during the day and then have abnormal blood pressure while asleep at night, which may be referred to as “nocturnal hypertension”. Such nocturnal hypertension disorders may be difficult to detect. For instance, typically, the blood pressure of a person may drop by 10 to 20 percent while sleeping which can give the endothelium and arteries time to recover and repair, while lowering the strain on the person's heart. Additionally, conditions such as sleep apnea may tend to periodically wake the person, which can not only increase a person's blood pressure, but also affect the person's sleep pattern and overall health. As another example, increased production of cortisol as morning approaches may also cause blood pressure spikes in some people, but, as mentioned above, these blood pressure spikes may be difficult to detect accurately.

To address these issues, some examples herein may include various sensors that may be positioned near, or adjacent to, the user to detect the position of the wearable device in relation to the ideal position for measuring the blood pressure of the user. Examples of such sensors may include a millimeter wave radar, camera, smart clothing, or other type of sensor that is able to detect the position of the wearable device while the blood pressure is being measured. Accordingly, rather than instructing the user to move the wearable device to the ideal location, the machine learning models may be trained to adjust the blood pressure measurement based on the various different positions that the wearable device may be located and detected throughout the night. As another example, the blood pressure measurement may be initiated only when the wearable device is detected as being within a threshold proximity to an ideal location for taking a blood pressure measurement. Numerous other variations will be apparent to those of skill in the art having the benefit of the disclosure herein.

FIG. 16 illustrates an example user interface 1600 that may be presented on the user device 104 before a user takes a spot blood pressure measurement according to some implementations. In this example, the user interface 1600 includes a recent blood pressure measurement history 1602, a selected recent blood pressure measurement 1604, and a recent pulse measurement 1606 corresponding to the selected recent blood pressure measurement 1604. For instance, the recent blood pressure measurement history 1602 may include a plurality of selectable recent blood pressure measurements 1608. The selected recent blood pressure measurement 1604 may include blood pressure information 1610 measured during the selected recent blood pressure measurement such as the systolic blood pressure and diastolic blood pressure of the user. In addition, the recent blood pressure measurement 1604 may include a confidence score 1611, as discussed above. Further, the recent blood pressure measurement 1604 may include a message or other indication 1612 as to whether there were any context factors indicated to have affected the blood pressure of the user at the time that the blood pressure measurement was taken. In this example, the indication 1612 indicates that there were no context factors present. Other examples in which context factors are present are discussed below.

The recent pulse measurement 1606 may include pulse information about the pulse of the user measured during the recent blood pressure measurement, and may show changes in pulse over time. When the user is ready to take another spot blood pressure measurement, the user may select a “Take BP Reading” button 1614, which may invoke the user interface of FIG. 17 discussed below.

Additionally, in this example, the user interface 1600 includes an “Add Context” button 1616 that the user may select to add context to the selected blood pressure measurement 1604. For instance, suppose that the selected blood pressure measurement 1604 is higher than usual for this user. The user may use the Add Context button 1616 to add one or more context factors, notes, or the like, that help explain why the blood pressure measurement is higher (or lower) than expected. Adding context may cause the message at 1612 to be changed to reflect the added context. Additional details of adding context are discussed below.

FIG. 17 illustrates an example user interface 1700 that may be presented on the user device 104 (or other suitable device) for receiving and presenting context information prior to beginning a blood pressure measurement according to some implementations. In this example, the user interface 1700 includes a listing of a plurality of context factors 1702 that may affect the user's blood pressure. For instance, the context factors 1702 included in the user interface 1700 would not necessarily affect the accuracy of the blood pressure measurement, but instead may affect at least temporarily (for a short term) the actual blood pressure of the user whose blood pressure is being measured. Any context factor(s) 1702 selected by the user may be associated with the corresponding blood pressure measurement, and may be referred to as a possible cause for an atypical temporary increase (or decrease) in the user's blood pressure during the corresponding blood pressure measurement.

In this example, the context factors 1702 include whether the user has exercised in the past hour 1704; whether the user has consumed a caffeinated drink, such as coffee, tea, energy drink, or the like, during the past three hours 1706; whether the user has taken blood pressure medication on the day of the measurement 1708; whether the user has eaten within the past two hours 1710; whether the user has consumed alcohol in the past 12 hours, whether the user has smoked in the past hour 1714; whether the user has taken a cold plunge or ice bath within the past three hours 1716; and whether the user has experienced menstruation within the past three hours 1718.

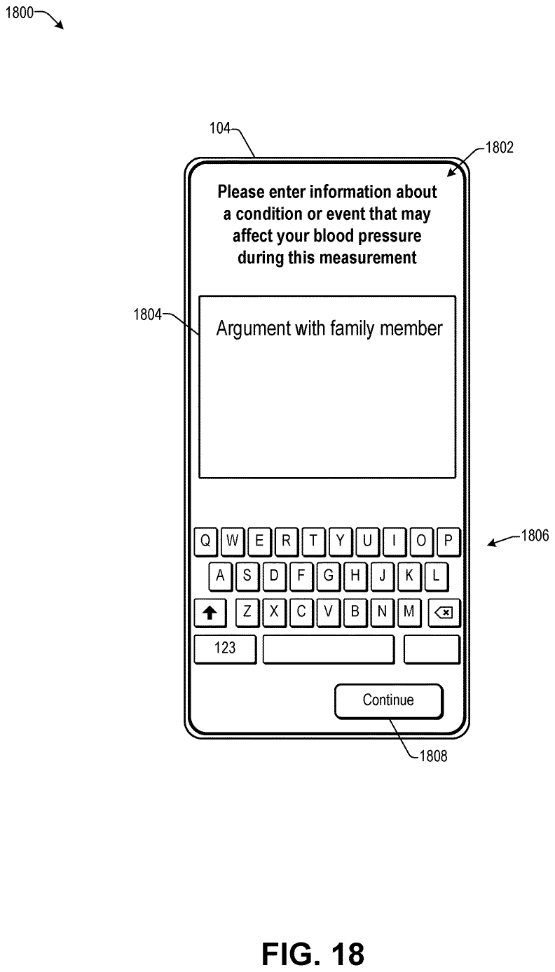

In addition, as indicated at 1720, the user interface may also provide the user with an opportunity to enter another context factor that the user may wish to record as possibly affecting the user's blood pressure, but which is not listed in the listing of selectable context factors 1704-1718. As discussed additionally below with respect to FIG. 18, an additional user interface may be presented when the user selects the option indicated at 1720 for enabling the user to enter another context factor not included in the listing of context factors 1702.

Furthermore, in some examples, one or more of the context factors may be automatically preselected for the user, such as based on input from one or more sensors on the user device and/or based on input from one or more other devices. As one example, the user may wear smart glasses that include one or more artificial intelligence (AI) programs executing thereon that may detect activities of the user in real time, and provide relevant activities to the user application 809 executing on the user device 104 (or alternatively to the wearable device 102) discussed above. For instance, the smart glasses may detect when the user exercises, may note that the user is consuming an energy drink, smoking, eating, drinking alcohol, and so forth, and may further note the time at which each of these events occurred. The user application 809 may receive this information from the smart glasses, such as through an application programming interface (API) or the like, and may prefill appropriate context factors 1704-1720 based on the information received from the smart glasses.

As another example, the user application on the user device may receive information from one or more applications on the user device for similarly automatically determining context factors that may apply to a particular blood pressure measurement. For example, the user may use a health application on the user device for tracking workouts, meals, beverage consumption, and so forth. One or more APIs may be used to automatically transfer relevant context information from the health application to the user application 809, and the user application 809 may prefill the appropriate context factors 1704-1720 automatically in response to receiving the information from the one or more applications executing on the user device 104.

As yet another example, one or more sensors onboard the user device 104 may also provide information related to one or more of the context factors 1702 to the user application 809. For example, an accelerometer and/or IMU may be used to detect that the user is exercising, and the user application may record the time and duration of the exercise. Based on this information, the user application 809 may preselect the exercise-within-past-hour context factor when the user accesses the user interface 1700. Furthermore, while several examples of automatically determining and applying context factors have been described above, numerous other techniques for detecting and applying relevant context factors to a blood pressure measurement will be apparent to those of skill in the art having the benefit of the disclosure herein.

Furthermore, the context factors 1702 are not limited to those illustrated, but may include any number of other context factors that may cause a measurable change in blood pressure. As one example, the listing of context factors 1702 may be scrollable in the up-down or left-right directions to view additional context factors 1702. The user may manually select one or more of the context factors 1702 that currently apply to the user, such as by tapping on, clicking on, or otherwise selecting one or more of the presented context factors 1704-1718. Alternatively, if the context factors have been selected automatically by the user application 809, the user may optionally verify the accuracy of the selected context factors. Following manual or automatic selection of one or more of the context factors 1702, the user may select a continue button 1722 to continue to a next user interface for performing the blood pressure measurement. As one example, the user interface 400 of FIG. 4, or the like, may be presented to start the blood pressure measurement procedure. Alternatively, if none of the context factors 1702 currently apply to the user, the user may select a “none apply” button 1724 to continue to a next user interface, such as the user interface 400 of FIG. 4.

FIG. 18 illustrates an example user interface 1800 that may be presented on the user device 104 according to some implementations. For example, the user interface 1800 may be presented when the user selects the option 1720 in the user interface 1700 of FIG. 17 for entering a context factor or other context information not included in the listing of context factors. As another example, the user interface 1800 may be presented when the user selects the add context button 1616 of FIG. 16 to add context to a blood pressure measurement that has already been taken.

In this example, the user interface 1800 includes instructions 1802, which instruct the user to enter information about a condition or event that the user believes may affect the user's blood pressure during the blood pressure measurement. A fillable text field 1804 may be included in the user interface 1800 for receiving a text entry to describe the event or condition that may be related to an atypical change in blood pressure of the user. Examples of such conditions or events may include an argument with a family member, an unexpected occurrence at work, or any of numerous other sources of stress or the like. Additionally, context factors that may lower blood pressure may also be added as context information. The added context information may be permanently associated with the corresponding blood pressure measurement.

A keyboard 1806 may be presented in the user interface 1800 to enable a user to enter the textual information describing the event or condition. Additionally, or alternatively, the user interface 1800 may enable the user to enter the information via a microphone or the like using speech-to-text conversion. When the user has finished entering the context information in the field 1804, the user may select a continue button 1808 to have the entry entered in the field 1804 associated with the current blood pressure measurement (in addition to any of the other context factors selected in the user interface 1700).

FIG. 19 illustrates an example user interface 1900 that may be presented on the user device 104 following completion of the blood pressure measurement according to some implementations. In this example suppose that the user selected, or the user application 809 had automatically detected that the user had consumed caffeine within the past three hours, had exercised within the past hour, and had provided another context factor, such as via the user interface 1800 as discussed above with respect to FIG. 18. Accordingly, in addition to presenting the measured systolic and diastolic blood pressure measurements, the user interface 1900 also may present one or more icons 1902 corresponding to the context factors that are indicated to be present during this blood pressure measurement. For instance, in this example, the icons include a caffeine icon Cf, an exercise icon E, and an “other context” icon O to visually indicate to the user which context factors are considered to have had an effect on the blood pressure of the user during this particular blood pressure measurement. In addition, the user interface 1900 may present the confidence score 1611 as discussed above e.g., with respect to FIG. 16. Furthermore, the user interface 1900 may present a message 1906 to the user to indicate that the blood pressure measurement may have been affected by the one or more context factors indicated at 1902, and accordingly to indicate to the user that the blood pressure measurement may be atypical due to the presence of the context factors and therefore not a diagnostic measurement.

In some cases, the user may wish to add more context to this blood pressure measurement, and may select the add context button 1616. Selection of the add context button 1616 may cause the application to present the user interface 1800 discussed above with respect to FIG. 18 for enabling the user to add additional comments or context on this particular blood pressure measurement.

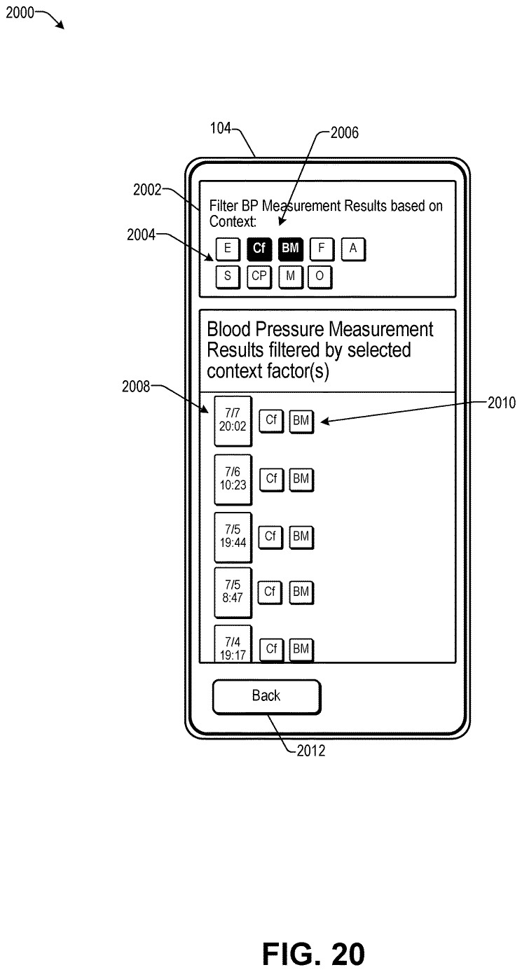

FIG. 20 illustrates an example user interface 2000 that enables the user to filter blood pressure measurement results based on context factors that may be associated with each blood pressure measurement according to some implementations. Thus the user interface 2000 may include a context factor selection area 2002 including a plurality of selectable context icons 2004. In this example, suppose that the user wishes to review all blood pressure measurements in which the user had consumed caffeine within three hours of the measurement, while also having taken blood pressure medication that day. Accordingly, the user has selected icons for caffeine Cf and blood pressure medication BM, as indicated at 2006. This selection results in a list 2008 of filtered blood pressure measurement results being presented along with the caffeine and blood pressure medication icons, as indicated at 2010. The user may scroll through the blood pressure measurement results, such as by scrolling up-down or left-right and may select one or more of the blood pressure measurement results to view the blood pressure information, pulse information, any additional context information, and the like. Further analysis options may be provided for, such as mean and average of all the presented blood pressure measurements, and the like. When the user has finished reviewing the filtered blood pressure measurement results, the user may select a back button 2012 to return to a previous user interface.

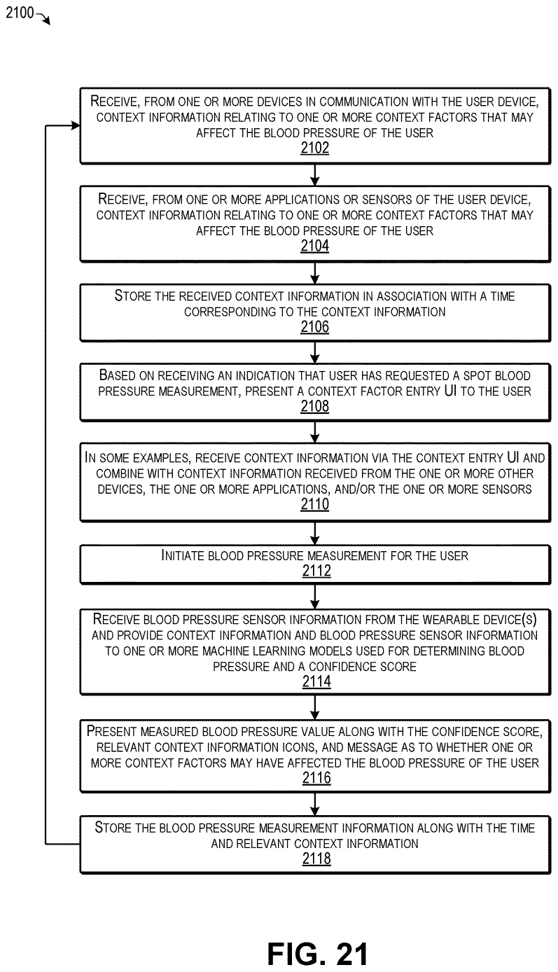

FIG. 21 is a flow diagram illustrating an example process 2100 for including context information with blood pressure measurements according to some implementations. In this example, the process 2100 may be executed as part of the user application executed on the user device 104, or may be part of a separate program, and/or may be executed on another device.

At block 2102, the device may receive, from one or more other devices in communication with the user device, context information related to one or more context factors that may affect the blood pressure of the user. For example, the user device may receive a communication from smart glasses or other wearable devices of the user regarding various activities of the user or other events that may affect the blood pressure of the user, such as consumption of caffeinated drinks, consumption of food, consumption of alcohol, exercise, smoking, or the like. As one example, an AI program may be active on the smart glasses or other wearable device of the user and may detect any food or drink that is consumed by the user, the time of the consumption, may detect exercise by the user, other activities of the user, and so forth, and may provide this information to the user application on the user device, such as via one or more APIs. For instance, in some examples, an AI associated with smart glasses may be able to maintain detailed records of the food and drink consumed by the user. Based on receiving this detailed information, the user application on the user device can associate particular food products and/or drinks with particular blood pressure measurements to enable identification of particular food or drinks that may cause increases or decreases in the blood pressure of the user. Specific activities detected by the smart glasses may similarly be associated with particular blood pressure measurements, thus providing the user with a comprehensive understanding of possible causes of atypical spikes in the blood pressure of the user. Accordingly, implementations herein are able to provide insights to particular activities, foods, drinks, etc., that may affect the blood pressure of the user in a manner that was not previously possible.

At block 2104, the device may receive, from one or more applications or sensors of the user device, context information related to one or more context factors that may affect the blood pressure of the user. For example, a health application, an exercise application, or the like, executing on the user device (or another device) may communicate with the user application via one or more APIs to provide information regarding exercise by the user, or other context information obtained by the health application, the exercise application, or other application regarding the user's activities. Additionally, or alternatively, one or more sensors on the user device may be used to detect when the user is exercising or participating in other activities that may affect the blood pressure of the user and the specific timing of these activities.

At 2106, the device may store the received context information in association with a time record corresponding to the context information. As one example, the context information may be stored only as long as it may affect the blood pressure of the user, and then may be overwritten or otherwise deleted.

At 2108, based on receiving an indication that the user has requested a spot blood pressure measurement, the device may present a context-factor-entry user interface to the user. For example, the device may present the user interface 1700 of FIG. 17 discussed above to enable the user to select one or more context factors that are relevant for a current blood pressure measurement. In some examples, one or more context factors may be automatically preselected by the user application. Further in some cases, the context factors may be entirely selected automatically, and the context entry user interface 1700 may not be used at all.

At 2110, in the case that the device receives context information entered manually via the context factor entry user interface 1700, the device may combine the context information received via the user interface with context information received from the one or more other devices, the one or more applications, and/or the one or more sensors. Accordingly, in some examples, the context information may include both context information entered manually by the user as well as context information that is detected by the user device or by other devices that the user may be wearing or that may be proximate to the user.

At 2112, the device may initiate blood pressure measurement for the user. For example, the user application on the user device may communicate via one or more APIs with the software executing on a wearable device of the user that includes one or more sensors for sensing blood pressure, such as the ring discussed above. In some examples, the process 700 discussed above with respect to FIG. 7 may be performed for determining a blood pressure measurement of the user.

At 2114, the device may receive blood pressure sensor information from the wearable device and may provide context information and the blood pressure sensor information to one or more machine-learning models used for determining blood pressure and a confidence score. Various examples of machine learning models that may be used for determining blood pressure and a corresponding confidence score are discussed above.

At 2116, the device may present a measured blood pressure value along with the confidence score, as well as relevant context information icons and a message as to whether one or more context factors may have affected the blood pressure of the user. For example, as discussed above with respect to FIG. 19, the device may present a user interface that includes one or more icons corresponding to one or more context factors that may have affected the blood pressure of the user during the corresponding blood pressure measurement.

At 2118, the device may store the blood pressure measurement information along with the time and relevant context information so that the user and/or the user application may refer to the stored information in the future.

The example processes described herein are only examples of processes provided for discussion purposes. Numerous other variations will be apparent to those of skill in the art in light of the disclosure herein. Further, while the disclosure herein sets forth several examples of suitable frameworks, architectures and environments for executing the processes, the implementations herein are not limited to the particular examples shown and discussed. Furthermore, this disclosure provides various example implementations, as described and as illustrated in the drawings. However, this disclosure is not limited to the implementations described and illustrated herein, but can extend to other implementations, as would be known or as would become known to those skilled in the art. Additionally, the implementations herein may be combined with any of the other implementations herein.

Various instructions, methods, and techniques described herein may be considered in the general context of computer-executable instructions, such as computer programs and applications stored on computer-readable media, and executed by the processor(s) herein. Generally, the terms program and application may be used interchangeably, and may include instructions, routines, modules, objects, components, data structures, executable code, etc., for performing particular tasks or implementing particular data types. These programs, applications, and the like, may be executed as native code or may be downloaded and executed, such as in a virtual machine or other just-in-time compilation execution environment. Typically, the functionality of the programs and applications may be combined or distributed as desired in various implementations. An implementation of these programs, applications, and techniques may be stored on computer storage media or transmitted across some form of communication media.

Although the subject matter has been described in language specific to structural features and/or methodological acts, it is to be understood that the subject matter defined in the appended claims is not necessarily limited to the specific features or acts described. Rather, the specific features and acts are disclosed as example forms of implementing the claims.

What is claimed:

1. A method comprising:
receiving, by one or more processors, context information determined automatically by at least one of a device, an application, or a sensor in communication with the one or more processors, wherein the context information corresponds to an event or condition that affects a blood pressure of a user of a wearable device;
receiving, by the one or more processors, photoplethysmographic (PPG) sensor data from the wearable device;
inputting, by the one or more processors, at least the received PPG sensor data into at least one computational model configured to output a blood pressure measurement based at least on the received PPG sensor data;
associating, by the one or more processors, the context information with the the blood pressure measurement; and
presenting, by the one or more processors, on a display in communication with the one or more processors, the blood pressure measurement and an indication of the context information associated with the blood pressure measurement.

2. The method as recited in claim 1, further comprising:
presenting, by the one or more processors, on the display, a listing of selectable context factors, wherein receiving the context information further includes receiving a selection of at least one selectable context factor in the user interface.

3. The method as recited in claim 1, further comprising:
presenting, by the one or more processors, on the display, a plurality of selectable indicators, each selectable indicator corresponding to a different context factor; and
based on receiving a selection of at least one of the selectable indicators, presenting, on the display one or more blood pressure measurement results associated with at least one context factor corresponding to the selected at least one selectable indicator.

4. The method as recited in claim 3, wherein the selectable indicators are icons presented on the display, each icon corresponding to a different type of context factor.

5. The method as recited in claim 1, wherein the device includes another wearable device configured to identify an activity or event corresponding to at least one context factor, the other wearable device configured to communicate, via an application programming interface, the identified activity or event to the one or more processors.

6. The method as recited in claim 1, wherein the application includes an application executed by the one or more processors and configured to identify an activity or event corresponding to at least one context factor, the application configured to communicate, via an application programming interface, the identified activity or event to a blood pressure application executable on the one or more processors for determining the blood pressure measurement.

7. The method as recited in claim 1, wherein the sensor includes at least one sensor in communication with the one or more processors, wherein an application executable on the one or more processors is configured to identify an activity or event corresponding to at least one context factor based on sensor information received from the sensor.

8. The method as recited in claim 1, wherein inputting, by the one or more processors, at least the received PPG sensor data into the at least one computational model further comprises inputting the context information into the at least one computational model.

9. The method as recited in claim 1, further comprising:
receiving, by the one or more processors, contemporaneously with the context information at least one of other sensor data or other information indicative of at least one of an environment of the user, a current or past activity of the user, or a bodily condition of the user; and
inputting, by the one or more processors, the at least one of the other sensor data or the other information into at least one computational model with the received PPG sensor data, wherein the at least one computational model is further configured to output a confidence score for the blood pressure measurement based at least in part on the least one of the other sensor data or the other information.

10. A method comprising:
receiving, by one or more processors, an indication to determine a blood pressure measurement of a user of a wearable device;
receiving, by the one or more processors, photoplethysmographic (PPG) sensor data from the wearable device;
receiving, by the one or more processors, at least one of other sensor data or other information indicative of at least one of an environment of the user, a current or past activity of the user, or a bodily condition of the user;
inputting, by the one or more processors, the received PPG sensor data and the at least one of the other sensor data or the other information into at least one computational model configured to output a blood pressure measurement and an associated confidence score based at least on the received PPG sensor data and the at least one of the other sensor data or the other information; and
based at least on the output of the at least one computational model including the blood pressure measurement and the associated confidence score, performing, by the one or more processors, at least one action.

11. The method as recited in claim 10, wherein the at least one computational model is a joint computational model configured to receive multiple different types of sensor data from multiple different types of sensors, respectively, and/or other types of information for estimating the blood pressure measurement and the associated confidence score.

12. The method as recited in claim 10, wherein the at least one computational model comprises at least a first dedicated model configured to receive the PPG sensor data as an input for determining the blood pressure measurement.

13. The method as recited in claim 12, wherein the at least one computational model further comprises at least one of:
a second dedicated model configured to receive the ECG sensor data as an input, or a third dedicated model configured to receive the BCG sensor data as an input,
the method further comprising aggregating an output of the first dedicated model with outputs of the at least one of the second dedicated model, or the third dedicated model to determine an aggregated blood pressure measurement.

14. The method as recited in claim 13, wherein the information used to determine the aggregated blood pressure measurement comprises a first set of information, the method further comprising applying a second set of one or more types of sensor data and/or other types of information from the at least one of the other sensor data or the other information for determining an associated confidence score for the aggregated blood pressure measurement.

15. The method as recited in claim 13, further comprising:
inputting the aggregated blood pressure measurement and remaining at least one of the sensor data or the other information into an optimized joint computational model that is configured to receive, as input the at least one of the other sensor data or the other information; and
receiving an output of the optimized joint computational model including the blood pressure measurement and associated confidence score.

16. The method as recited in claim 10, further comprising:
determining, based at least on the sensor data, an indication of at least one physiological characteristic of a portion of a vascular system of the user; and
providing the at least one physiological characteristic as an input to the at least one computational model.

17. The method as recited in claim 10, wherein in addition to the PPG sensor data, the at least one of the other sensor data or the other information includes at least one of: electrocardiogram sensor data; ballistocardiographic (BCG) sensor data; sound information; inertial measurement unit (IMU) and/or motion information; device usage information; user posture and/or user position information; user recent physical activity information; user phone and/or personal computer (PC) sensor data; user phone and/or PC software information; other user wearable device information; or Internet of things (IoT) device data.

18. The method as recited in claim 10, wherein the at least one computational model is a joint computational model configured to receive a first set of one or more types of sensor data from a first set of one or more sensors, respectively, for determining the blood pressure measurement, the at least one computational model further configured to apply a second set of one or more types of sensor data or other types of information from the at least one of the other sensor data or the other information for determining the associated confidence score for the blood pressure measurement.

19. The method as recited in claim 10, further comprising:
receiving contemporaneously with the at least one of other sensor data or other information, context information determined automatically by at least one of a device, an application, or a sensor in communication with the one or more processors, wherein the context information corresponds to an event or condition that affects a blood pressure of a user of a wearable device; and
at least one of:
inputting the context information into at least one computational model; or
associating the context information with the blood pressure measurement.

20. A device comprising:
one or more processors configured by executable instructions to perform operations comprising:
receiving, by the one or more processors, an indication of a user input indicative of performing a measurement of a bodily condition;
receiving, by the one or more processors, an indication of at least one of a position of a body of a user or a position of a wearable device relative to the body of the user;
determining, by the one or more processors, whether the at least one of the position of the body of the user or the position of the wearable device relative to the body of the user is within a threshold of optimal for performing measurement of the bodily condition;
based at least on determining that the at least one of the position of the body of the user or the position of the wearable device relative to the body of the user is not within the threshold of optimal for performing the measurement of the bodily condition, causing, by the one or more processors, presentation in real time of at least one instruction to the user to reposition the at least one of the body of the user or the position of the wearable device relative to the body of the user; and
causing the measurement of the bodily condition to be performed based at least on presenting the at least one instruction.
